(19) United States
(12) Patent Application Publication
(10) Pub. No.: US 2012/0107229 A1
(43) Pub. Date: May 3, 2012

(54) NOVEL NANO-PROBES FOR MOLECULAR IMAGING AND TARGETED THERAPY OF DISEASES

(76) Inventors: Xuefei Huang, Okemos, MI (US); David Zhu, East Lansing, MI (US); George Abela, East Lansing, MI (US)

(21) Appl. No.: 13/264,823

(22) PCT Filed: Apr. 14, 2010
(86) PCT No.: PCT/US2010/031064

§ 371 Date: Jan. 17, 2012

Related U.S. Application Data
(60) Provisional application No. 61/169,456, filed on Apr. 15, 2009.

(51) Int. Cl. A61K 49/00; A61K 49/04; A61K 49/18; A61P 9/10; A61K 31/366; A61K 31/655; A61K 51/12; A61K 31/713; B82Y 5/00

(52) U.S. Cl. 424/1.11; 424/9.4; 424/9.32; 424/9.6; 424/9.3; 514/44 A; 514/451; 514/150; 977/773; 977/928; 977/930; 977/927; 977/906

(57) ABSTRACT

The present inventions relate to compositions and methods for imaging and treating atherosclerotic diseases, pathogen infections, and tumors by administering actively targeting magnetic nanoparticles. In particular, the present inventions provide new types of targeting ligands attached to magnetic nanoparticles for magnetic resonance imaging. The use of these targeted magnetic nanoparticles is contemplated as a means to treat atherosclerotic diseases, including but not limited to inhibiting and removing atherosclerotic plaques. Further, actively targeting magnetic nanoparticles are contemplated for use with multiple labels for use in nuclear medicine imaging, computed tomography (CT) techniques and other types of imaging for medical and research applications.

### FIELD OF THE INVENTION

The present inventions relate to compositions and methods for imaging and treating atherosclerotic diseases, pathogen infections, and tumors by administering actively targeting magnetic nanoparticles. In particular, the present inventions provide new types of targeting ligands attached to magnetic nanoparticles for magnetic resonance imaging. The use of these targeted magnetic nanoparticles is contemplated as a means to treat atherosclerotic diseases, including but not limited to inhibiting and removing atherosclerotic plaques. Further, actively targeting magnetic nanoparticles are contemplated for use with multiple labels for use in nuclear medicine imaging, computed tomography (CT) techniques and other types of imaging for medical and research applications.

### BACKGROUND OF THE INVENTION

Atherosclerotic diseases are the leading causes of morbidity and mortality in the Western world. Atherosclerotic plaques, one of the primary causes of acute cardiac events, are formed through accumulation of white blood cells (e.g. macrophages and T lymphocytes), lipids (e.g. cholesterol), and fibrous connective tissue underneath the endothelium lining in artery walls. Plaques form when an area of an artery becomes inflamed in a manner to trigger lipid and macrophage accumulation. While plaques are found with a variety of morphology many of these plaques are labeled “unstable.” These unstable plaques often referred to as vulnerable plaques, have high tendency to rupture, subsequently blocking blood vessels leading to heart attacks and strokes. During the past decade, although lipid-lowering therapies have demonstrated direct clinical benefits to cardiovascular diseases, they have only reduced acute cardiac events by 30 percent. Further, there is a lack of diagnostic methods to detect, monitor and treat atherosclerotic plaques.

Therefore there is a need for improved compositions and methods to identify atherosclerotic plaques in patients at risk for atherosclerotic disease at an early stage in order to slow or prevent plaque formation, to improve the efficacies of therapeutic agents and to noninvasively assess the benefits of therapies intended to extend life spans atherosclerotic patients.

### SUMMARY OF THE INVENTION

The present inventions relate to compositions and methods for imaging and treating atherosclerotic diseases, pathogen infections, and tumors by administering actively targeting magnetic nanoparticles. In particular, the present inventions provide new types of targeting ligands attached to magnetic nanoparticles for magnetic resonance imaging. The use of these targeted magnetic nanoparticles is contemplated as a means to treat atherosclerotic diseases, including but not limited to inhibiting and removing atherosclerotic plaques. Further, actively targeting magnetic nanoparticles are contemplated for use with multiple labels for use in nuclear medicine imaging, computed tomography (CT) techniques and other types of imaging for medical and research applications.

The inventions provide a composition, comprising a targeting ligand, wherein said targeting ligand targets a molecule associated with an atherosclerotic plaque, and a nanoparticle. In one embodiment, said targeting ligand is selected from the group consisting of hyaluronic acid, dextrin, cyclodextrin, mannose, a lectin, siRNA, MMP-9 sensitive linker, MMP-9 substrate peptide SGPLF, statin, fibrin, NO prodrug, Simivastatin, selectins and CD44 structural analogs. In one embodiment, said targeting ligand is selected from the group consisting of a hyaluronic acid (HA) molecule and a cyclodextrin (CD) dimer. In one embodiment, said molecule is a target analyte. In one embodiment, said target analyte is selected from the group consisting of CD44, Vascular Cell Adhesion Molecule-1, Inter-Cellular Adhesion Molecule-1, cholesterol, fibrin, MMP, and phosphatidyl serine. In one embodiment, said target analyte is selected from the group consisting of CD44 and cholesterol. In one embodiment, said nanoparticle further comprises a therapeutic agent. In one embodiment, said therapeutic agent is selected from the group consisting of a siRNA, an anti-proinflammatory agent, interleukins, growth factors, siRNA, MMP inhibitors, cholesterol binding molecules, cortisone steroids and a HA antiadhesive. In one embodiment, said therapeutic agent is selected from the group consisting of lovastatin, diazeniumdiolate, anti-TNF-α siRNA, interferon (IFN)-γ, a colony stimulating factors, and a MMP-9 sensitive linker. In one embodiment, said nanoparticle comprises a magnetic compound. In one embodiment, said magnetic compound is selected from the group consisting of a Fe₃O₄ and iron-platinum (FePt). In one embodiment, said nanoparticle further comprises a positron emission tomography (PET) label capable of providing a diagnostic computed tomography (CT) image, wherein said PET label is selected from the group consisting of PET tracer ⁶⁴Cu and 18F labeled 2-deoxy glucose (18FDG). In one embodiment, said nanoparticle further comprises a molecule capable of providing a nuclear magnetic resonance (NMR) image, wherein said molecule is gadolinium. In one embodiment, said nanoparticle is capable of providing a magnetic resonance spectroscopy (MRS) spectrum. In one embodiment, said nanoparticle has a diameter less than or equal to 6 nm. In one embodiment, said nanoparticle further comprises a fluorophore. In one embodiment, said nanoparticle further comprises a fluorescein isocyanate (FITC) molecule. In one embodiment, said nanoparticles further comprise a modifying molecule. In one embodiment, said modifying molecule is selected from the group consisting of dextran, epichlorohydrin, diazeniumdiolate, hydrazide groups, PEG, and a hexasaccharide attached to an azide molecule. In one embodiment, said nanoparticle has a core and a shell. In one embodiment, said nanoparticle core comprises a magnetic compound. In one embodiment, said magnetic compound is superparamagnetic. In one embodiment, said nanoparticle is water-soluble. In one embodiment, said is water soluble inside of a blood vessel. In one embodiment, said shell comprises dextran. In one embodiment, said shell is dextran. In one embodiment, said shell comprises silica. In one embodiment, said nanoparticle is selected from the group consisting of Iron Oxide, Fe₃O₄, iron cobalt, gold and iron-platinum (FePt). In one embodiment, said nanoparticle is selected from the group consisting of superparamagnetic iron oxide nanoparticle (SPION), dextran-epichlorohydrin nanoparticle (DESPION), amine coated nanoparticle (DSPION), ammonia treated dextran-epichlorohydrin nanoparticle (DESPION-NH2). In one embodiment, said nanoparticle further comprise a bifunctional linker, wherein a bifunctional linker is a PEG-3000 conjugate.

The inventions provide a method, comprising, a) providing, i) a composition comprising a targeting ligand, wherein said targeting ligand targets a molecule associated with an atherosclerotic plaque, and a nanoparticle, and ii) a patient at risk for a cardiac event; and administering said particle to said patient for providing a diagnostic image of an atherosclerotic plaque. In one embodiment, said targeting ligand is selected from the group consisting of a hyaluronic acid (HA) molecule and a cyclodextrin (CD) dimer. In one embodiment, said targeting ligand binds to a target analyte. In one embodiment, said targeting ligand alters a target analyte. In one embodiment, said alters is lowing the activity of a target analyte. In one embodiment, said hyaluronic acid (HA) molecule hits a plaque macrophage. In one embodiment, said cyclodextrin (CD) dimer hits a cholesterol crystal. In one embodiment, said nanoparticle further comprises a therapeutic agent for targeted drug delivery. In one embodiment, said nanoparticle is capable of delivering the therapeutic agent to a plaque; wherein said therapeutic agent is selected from the group consisting of a siRNA, a MMP-9 sensitive linker, MMP-9 substrate peptide SGPLF, a statin, lovastatin, fibrin, a nitrix oxide (NO) prodrug, Simivastatin™, selectins, CD44 structural analogs, anti-proinflammatory agent, interleukin, growth factor, siRNA of TNF-alpha, MMP inhibitor, cholesterol binding molecule, paclitaxel, cortisone steroid and a HA antiadhesive. In one embodiment, said targeting ligand targets a physiological event selected from the group consisting of macrophage activation, cholesterol crystal formation, angiogenesis, and apoptosis. In one embodiment, said patient has atherosclerosis. In one embodiment, said atherosclerotic plaque is selected from the group consisting of a pre-plaque, wherein a pre-plaque is a damaged artery wall, a vulnerable plaque, and a growing plaque. In one embodiment, said nanoparticle further comprises a compound capable of providing a medical image, wherein said image is provided by devices selected from the group consisting of magnetic resonance spectroscopy (MRS), nuclear magnetic resonance imaging (NMR), multimodal imaging, fluorescent, positron emission tomography (PET) and computed tomography (CT). In one embodiment, said administering is injecting 1-180 micromol/kg of said nanoparticle. In a preferred embodiment, said administering is injecting 50-60 micromol/kg of said nanoparticle. In one embodiment, said administering is injecting a concentration of 0.0001-5000 mg Fe/mL. In a preferred embodiment, said administering is injecting a concentration of 0.05 mg Fe/mL. In one embodiment, said nanoparticle has a core and a shell. In one embodiment, said nanoparticle core comprises a magnetic compound. In one embodiment, said magnetic compound is superparamagnetic. In one embodiment, said nanoparticle is water-soluble. In one embodiment, said nanoparticle is soluble inside of a blood vessel. In one embodiment, said shell comprises dextran. In one embodiment, said shell is dextran. In one embodiment, said shell comprises silica. In one embodiment, said nanoparticle is selected from the group consisting of Iron Oxide, Fe₃O₄, iron cobalt, gold and iron-platinum (FePt). In one embodiment, said nanoparticle is selected from the group consisting of superparamagnetic iron oxide nanoparticle (SPION), dextran-epichlorohydrin nanoparticle (DESPION), amine coated nanoparticle (DSPION), ammonia treated dextran-epichlorohydrin nanoparticle (DESPION-NH₂). In one embodiment, said nanoparticle further comprise a bifunctional linker, wherein a bifunctional linker is a PEG-3000 conjugate.

The inventions provide a method, comprising, a) providing, i) a composition, comprising a targeting ligand, wherein said targeting ligand targets a molecule associated with an atherosclerotic plaque, a therapeutic agent, and a nanoparticle; ii) a patient at risk for a cardiac event; iii) a magnetic resonance imaging device; and b) administering said nanoparticle to said patient for providing a benefit to a patient. In one embodiment, said benefit is reducing the numbers of plaques. In one embodiment, said benefit is reducing the size of a plaque. In one embodiment, said benefit is slowing the growth of a plaque. In a preferred embodiment, said benefit is increasing the life-span of a patient over an untreated patient.

### DEFINITIONS

The use of terms defined in the singular are intended to include those terms defined in the plural and vice versa. The use of the article “a” or “an” is intended to include one or more.

As used herein, “atherosclerotic disease” “atherosclerotic diseases” refers to a syndrome affecting arterial blood vessels involving a chronic inflammatory response within and on the walls of arteries forming plaques within the arteries and arterol walls. It is commonly referred to as a hardening or furring of the arteries.

As used herein, “atherosclerotic plaque” or “plaque” refers to an accumulation of white blood cells (such as macrophages, and other white blood cells such as T lymphocytes, etc.), extracellular matrix, lipids (cholesterol and lipids), cholesterol crystal, and fibrous connective tissue underneath the endothelium lining in artery walls. The term “vulnerable plaque” in reference to an atherosclerotic plaque refers to an unstable plaque known for a high tendency to rupture, subsequently blocking blood vessels leading to a heart attack and strokes. The term “pre-plaque” refers to an area of damaged artery wall, for example, an arterial wall showing inflammatory characteristics. The term “growing plaque” refers to an inflammatory response resulting in enlarging the plaque.

As used herein, “a molecule associated with an atherosclerotic plaque” refers to any molecule attached to a plaque, such as a molecule as part of the extracellular matrix, on the surface of a cell attached to a plaque, a molecule associated with plaque formation, a molecule associated with a physiological event associated with plaque formation and the like. For use herein, a molecule associated with an atherosclerotic plaque is also a “target” for a magnetic nanoparticle of the present inventions such that a “targeting ligand” “targets” or “targeted” a molecule associated with an atherosclerotic plaque. The actual target may be a plaque associated molecule located in the extracellular matrix of a plaque, or associated with plaque formation, such as carbohydrates, lipids, e.g. LDL, proteins, and the like. Preferably, the target cell is present on the cell surface of an arterial endothelial cell or macrophage associated with a plaque e.g., dysfunction or damaged endothelial cells.

As used herein, in general a molecule is “targeted” when a nanoparticle binds to or is taken up by a target cell, (e.g., an activated macrophage cell, vascular endothelial cell, etc.), or alters a target molecule, (e.g. a nanoparticle comprising an enzyme, i.e. MMP, for altering a substrate), or when a nanoparticle that “hits” a cholesterol crystal, a nucleotide (e.g. TNF-alpha, and the like). For example, a “targeted” cell may increase or decrease the level of expression, activity, half-life and the like of an enzyme or gene, (e.g., the use of siRNA targeting TNF-alpha for decreasing the expression of TNF-alpha) and the like.

As used herein, the terms “ligand” and “ligand molecule” refer to any molecule that is able to bind to another molecule (e.g. a target, a target analyte, and the like). Preferably, the ligand molecules of the present invention are displayed on the surface (e.g. attached to the nanoparticle in a manner such that the ligand may attaché to its target, be accessible as a substrate for an enzyme, be accessable to act upon a substrate, etc.) of a magnetic nanoparticle (e.g. covalently attached via a liker to a magnetic nanoparticle). Example of ligand molecules include, but are not limited to, carbohydrates, (i.e. multimers of haluronic acid units, proteins (e.g. enzymes, receptors, etc.), lipids, or nucleic acids.

The term “targeting ligand” refers to a molecule, such as a carbohydrate, lipid, peptide, protein, glycoprotein, etc., that are used in conjunction with nanoparticles of the present invention in order to “target” a molecule, such as a cell surface marker, e.g., surface CD44 of a macrophage, a dysfunctional endothelial cell or even a cell that naturally or artificially is expressing a cell surface marker to which the targeting ligand binds, or for targeting a molecule involved with a pathway associated with a plaque, such as an MMP substrate for activating MMP for decreasing plaque formation, or for hitting a physiological event or for removing a plaque.

As used herein, the term “target analyte” refers to a molecule to be detected or targeted by the targeting ligand attached to magnetic nanoparticles. Examples of target molecules include, but are not limited to, cells in a subject, pathogens, such as bacteria and viruses, antibodies, naturally occurring drugs, synthetic drugs, pollutants, allergens, effector molecules, growth factors, chemokines, cytokines, and lymphokines. Preferably, the target analysts are found within blood vessels, such as in blood plasma (e.g. fibrin in human blood plasma), on the surface of circulating cells, on the surface of cells involved with plaque formation (i.e. CD44), on the surface of cells within plaques, or on the surface of blood vessel walls, as part of extracellular matrix of blood vessel lumen, extracellular matrix of an atherosclerotic plaque, and the like.

As used herein, the term “active targeting” refers to methods of the present inventions for providing a nanoparticle “hit” to a specific target as opposed to “passive targeting” which refers to nanoparticles used in methods for nonspecific take-up by cells and tissues, such as nanoparticles in the size range for triggering pinocytotic take up or accumulation in a range of tissues or cells or nonreceptor mediated phagocytosis.

As used herein, “target” as a general reference to a “biological target” refers to an organism, cell, microorganism, bacteria, virus, fungus, plant, prion, protozoa, or pathogen or portion of an organism, cell, microorganism, bacteria, virus, fungus, plant, prion, protozoa or pathogen for use in imaging or desired for removal. A target includes a tissue, a biological pathway, such as blood clotting, or a biological phenomenon, such as arterial wall damage, arterial inflammation, plaque formation, etc.

As used herein, the term “haluronic acid” or “hyaluronan” or “hyaluronate” refers to a non-sulfated glycosaminoglycan polymer of disaccharides, composed of D-glucuronic acid and D-N-acetylglucosamine, linked together via alternating β-1,4 and β-1,3 glycosidic bonds.

As used herein, the term “CD” or “cluster of differentiation” refers to a molecule or “marker” found on the surface of a cell. When CD is shown with a number, such as “CD44” the number refers to a particular type of molecule as assigned using guidelines following “CD nomenclature” established by the 1^{st} International Workshop and Conference on Human Leukocyte Differentiation Antigens (HLDA). Specifically, the term “CD44” refers to a molecule expressed by the CD44 gene, such that “CD44” or “CD44 antigen” may represent a number of isoforms from one species, including alternatively spliced variants of CD44. In general, CD44 refers to molecule that mediates cell-cell and cell-matrix interactions in part through its receptor affinity for binding a ligand, such as hyaluronic acid (HA), osteopontin, collagens, and matrix metalloproteinases (MMPS), and the like. CD44 homologs, i.e. amino acid sequences within 50% identity to human CD44 proteins (including fragments), are found in chimpanzee, several great ape species, rats, mice, and cows.

As used herein, the term “structural analogs” or “structural analogues” or “chemical analogs” or “analogs” refer compounds in which one or more atoms, functional groups, or substructures have been replaced with different atoms, groups, or substructures, for example, a CD44 analog, etc.

As used herein, the term “dextrin” refers to a group of low-molecular-weight carbohydrates produced by the hydrolysis of starch.

As used herein, the term “dextran” refers to a complex, branched glucan (polysaccharide made of many glucose molecules) composed of chains of varying lengths (complex ranging from 10 to 150 kilodaltons).

As used herein, the term “cyclodextrin” or “cycloamylose” refers to a family of cyclic oligosaccharides, composed of at least 5 or more α-D-glucopyranoside units linked 1->4, as in “amylose” a fragment of starch.

As used herein, the term “Man” or “mannose” refers to a sugar monomer of the aldohexose series of carbohydrates

As used herein, the term “lectin” refers to a sugar-binding protein which is highly specific for a specific sugar moietie, for example, a lectin of mannose binds specifically to “Concanavalin A” or “CON A.”

As used herein, the term “fibrin” or “Factor Ia” refers a fibrous protein involved in the clotting of blood. Fibrin is made from fibrinogen, a soluble plasma glycoprotein that is synthesised by the liver. Processes in the coagulation cascade activate the zymogen prothrombin to the serine protease thrombin, which is responsible for converting fibrinogen into fibrin. Fibrin is then cross-linked by factor XIII to form a clot.

As used herein, the term “selectin” or “CAM” or “cell adhesion molecule” refers a family of single-chain transmembrane glycoproteins that share properties similar to C-type lectins due to a related amino terminus and calcium-dependent binding. Selectins bind to sugar moieties and so are considered to be a type of lectin, in other words, a cell adhesion protein that binds to a sugar polymer.

As used herein, the term “matrix metalloproteinase” or “MMP” refers an enzyme which may be an individual enzyme up to a family of matrixin enzymes.

As used herein, the term “matrix metalloproteinase 9” or “MMP-9” or “gelatinase B” refers a member of the matrixin family of metalloendopeptidases that is capable of cleaving gelatin, a denatured form of collagen.

As used herein, the term “MMP-9 sensitive linker” refers a molecule capable of being cleaved by MMP-9.

As used herein, the term “MMP-9 substrate peptide” refers a peptide capable of activating MMP-9, for example, SGPLF.

As used herein, the term “nitric oxide” or “nitrogen monoxide” refers to a chemical compound with chemical formula NO.

As used herein, the term “nitric oxide prodrug” or “NO prodrug” refers to a therapeutic agent for slowing plaque growth or reducing plaque size, for example, blocking apoptosis of plaque macrophages.

As used herein, the term “prodrug” refers to a masked form of an active drug or an inactive drug, designed to be activated once it's administered, such as asprin. For the purposes of the present inventions, a produg refers to an active form released when an enzyme or physiological process removes a part or group from the prodrug revealing an active form of the drug

As used herein, the term “binding” refers to an attachment that occurs between a paired species such as a carbohydrate to a receptor, e.g. CD44 and HA, enzyme/substrate, receptor/agonist, antibody/antigen, and lectin/carbohydrate, which may be mediated by covalent or non-covalent interactions or a combination of covalent and non-covalent interactions. When the interaction of two species produces a non-covalently bound complex, the binding that occurs is typically electrostatic, hydrogen-bonding or the result of lipophilic interactions. Accordingly, “specific binding” occurs between a paired species that produces a bound complex having the binding and/or specificity characteristics of a receptor/ligand, antibody/antigen or enzyme/substrate interaction. For example, the specific binding is characterized by the binding of one member of a pair to a particular species and to no other species within the family of compounds to which the corresponding member of the binding member belongs. Thus, for example, a ligand such as HA binds preferably to a unique receptor, such as CD44, an antibody binds preferably to a unique epitope, and the like.

As used herein, the term “delivering” or “administration” in reference to a nanoparticle of the present inventions refers to the placement of nanoparticles at, next to, or sufficiently close to a location containing a target, e.g., intravenously, in order to maximize the number of particles that will be able to contact targets at the target location within the luman of an artery. Delivering may also refer to adding a nanoparticle to a cell culture in vitro or injecting a nanoparticle into a tissue or organ in vivo.

The administration of a nanoparticle of the present inventions preferably reverses or ameliorates atherosclerotic disease such that reduction of acute cardiac events associated with the presence of an atherosclerotic plaque is preferably at least reduced statistically by 30%, 40%, 50%, 60%, 70%, 80%, 90%, or ideally near or at 100% (such that no cardiac event occurs).

As used herein, “administration of a nanoparticle” of the present inventions preferably reverses or ameliorates plaques such that damage to arterial walls caused by inflammatory cells, cholesterol crystal or other related disease conditions is reduced preferably by at least 20%, 30%, 40%, 50%, 60%, 70%, 80%, 90%, or ideally near or at 100% (such that a previously imaged plaque is nearly or completely removed). The reduction of plaque size can be assessed by MRI. Arteriol damage can be assessed by MRI.

As used herein, the term “therapeutic” or “therapeutic agent” refers to any compound that provides a benefit to a patient, such as a therapeutic agent attached to an active nanoparticle of the present inventions, for example, siRNA encoding a silencing DNA sequence for TNF-alpha.

As used herein, the term “patient” refers to a human or animal and need not be hospitalized. For example, out-patients, persons in nursing homes are “patients.”

As used herein, the term “cardiac event” includes but is not limited to blocked blood vessels, heart attacks and strokes.

As used herein, the term “patient at risk for a cardiac event” includes, but is not limited to, patients with plaques, patients with high blood pressure, trauma patients, intensive care patients, intubated patients, elderly patients, low birth weight patients and immunocompromised patients.

As used herein, the term “statin” or “HMG-CoA reductase inhibitor” in reference to a therapeutic refers to a class of drugs that lower cholesterol levels in people with or at risk of cardiovascular disease, for example, medications named Levacor, Zocor, Pravachol, Lipitor, Crestor, and the like and compounds, such as lovastatin, including synthetic compounds, such as “Zocor™” or “Simvastatin™, where Simvastatin refers to a synthetic derivate of a fermentation product of Aspergillus terreus,” etc.

As used herein, “lovastatin” refers to a specific inhibitor of three-hydroxy-3-methylglutaryl coenzyme A (HMG-CoA) reductase.

As used herein, the term “targeted delivery of a contrast agent” or “targeted delivery of a nanoparticle” of the present inventions refers to delivering an agent or drug to the area of a plaque or to a damaged arterial wall.

As used herein, the term “targeted delivery of a therapeutic agent” refers to refers to “delivering” or “releasing” a therapeutic agent directly to a plaque or at the edge of a plaque or to a cell associated with a plaque or a damaged arterial wall that is likely to lead to plaque formation.

As used herein, the term “nanoparticle” refers to a small particle (e.g. nanometer range). Even though particles can be of any size, the preferred size is 0.1-500 nanometers, preferably 1-150 nanometers, more preferably 5-15 nanometers, and most preferably about 6 nanometers. The particles may be uniform (e.g., being about the same size) or of variable size. Particles may be any shape (e.g. spherical or rod shaped), but are preferably made of regularly shaped material (e.g. spherical). Examples of such nanoparticles are shown in Palmacci, et al., Synthesis of Polysaccharide Covered Superparamagnetic Oxide Colloids Patent WO/1995/005669, 1995, herein incorporated by reference. A nanoparticle may have a core and be covered by a shell.

As used herein, “core” in reference to a nanoparticle refers to the center of a nanoparticle. A core may also be the primary material of a nanoparticle, for example, a magnetic material for the core of a nanoparticle.

As used herein, “shell” in reference to a nanoparticle refers to a molecule that encapsulates or surrounds a nanoparticle core. A shell is at least one type of molecule or layer and a core may have one or more shells that generally surround at least a portion of one core. Several cores may be incorporated into a larger nanoshell.

As used herein, “fluorophore” refers to a compound that under certain conditions emits a fluorescent signal, such as fluorescein isothiocyanate (FITC) or derivative thereof (including those available from Molecular Probes, Inc.).

As used herein, “magnetism refers to a phenomena by which materials exert attractive or repulsive forces on other materials. Some well-known materials that exhibit easily detectable magnetic properties (called magnets) are nickel, iron, cobalt, gadolinium and their alloys; however, all materials are influenced to greater or lesser degree by the presence of a magnetic field

As used herein, the term “magnetic” refers to the capability of responding to a magnetic field.

As used herein, “magnet” or “Magnesian stone” refers to a material or object that produces a magnetic field.

As used herein, “magnetic field” refers to a vector field which surrounds magnets and electric currents, and which is detected by virtue of the fact that it exerts a force on moving electric charges and on magnetic materials.

As used herein, “Paramagnetism” refers to a form of magnetism which occurs only in the presence of an externally applied magnetic field, such as applied by an MRI machine.

As used herein, “Superparamagnetism” refers to a form of magnetism. A superparamagnetic material is composed of small ferromagnetic clusters (e.g. crystallites), but where the clusters are so small that they can randomly flip direction under thermal fluctuations. As a result, the material as a whole is not magnetized except in an externally applied magnetic field (in that respect, it is like paramagnetism). Unlike ferromagnets, paramagnets do not retain any magnetization in the absence of an externally applied magnetic field, thus paramagnetic materials rapidly lose magnetism when the MRI magnetic field is reduced or turned off. In a preferred embodiment, nanoparticles are paramagnetic.

As used herein, “magnetic moment” in reference to a system (such as a loop of electric current, a bar magnet, an electron, a molecule, or a planet) usually refers to its magnetic dipole moment, and is a measure of the strength of the system's net magnetic source.

As used herein, “magnetic dipole moment” refers to a magnetic moment of an electron caused by its intrinsic property of spin

As used herein, “magnetic resonance imaging” or “MRI” or “nuclear magnetic resonance imaging” or “NMRI” refers to a medical imaging technique most commonly used in radiology to visualize the internal structure and function of the body. MRI provides much greater contrast between the different soft tissues of the body than computed tomography (CT). Unlike CT, it uses a powerful magnetic field to align the nuclear magnetization of (usually) hydrogen atoms in water in the body. Radio frequency (RF) fields are used to systematically alter the alignment of this magnetization, causing the hydrogen nuclei to produce a rotating magnetic field detectable by the scanner. This signal can be manipulated by additional magnetic fields to build up enough information to construct an image of the body

As used herein, the term “magnetic nanoparticle” refers to a nanoparticle that is magnetized by a magnetic field, for example, capable of providing a clear image of a specific tissue, plaque, or organ in a body.

As used herein, “medical imaging” refers to the techniques and processes used to create images of the human body (or parts thereof) for clinical purposes (medical procedures seeking to reveal, diagnose or examine disease) or medical science (including the study of normal anatomy and physiology).

The term “sample” in the present specification and claims is used in its broadest sense.

As used herein, “biological samples” refers to animal, including human, fluid, solid (e.g., plasma) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc. “Environmental samples” include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

As used herein, the term “molecular recognition element” refers to molecules capable of specifically (i.e., non-randomly) binding to, hybridizing to, or otherwise interacting with a desired target molecule. Examples of molecular recognition elements include, but are not limited to, nucleic acid molecules (e.g., RNA and DNA, including ligand-binding RNA molecules), polypeptides (e.g., antigen binding proteins, receptor ligands, signal peptides, hydrophobic membrane spanning domains), antibodies (and portions thereof), organic molecules (e.g., biotin, carbohydrates, glycoproteins), and inorganic molecules (e.g., vitamins). A given drug delivery composition may have affixed thereto one or a variety of molecular recognition elements.

As used herein, the term “membrane receptors” refers to constituents of membranes that are capable of interacting with other molecules or materials. Such constituents can include, but are not limited to, proteins, lipids, carbohydrates, and combinations thereof.

As used herein, the term “carbohydrate” refers to a class of molecules including, but not limited to, sugars, starches, cellulose, chitin, glycogen, and similar structures. Carbohydrates can also exist as components of glycolipids and glycoproteins.

As used herein, the term “linker” or “spacer molecule” refers to material that links one entity to another. In one sense, a molecule or molecular group can be a linker that is covalently attached two or more other molecules (e.g., linking a ligand to a self-assembling monomer).

As used herein, the term “bifunctional,” refers to a linker molecule with two functional groups that react with different chemical groups (e.g., primary amines, esters or aledehydes).

As used herein, the term “epichlorohydrin” refers to any of the chiral molecules comprising an organochlorine compound and an epoxide (e.g. CAS Number: 106-89-8).

As used herein, the term “covalent bond” refers to the linkage of two atoms by the sharing of at least one electron, contributed by each of the atoms.

As used herein, the term “cell culture” refers to any in vitro culture of cells. Included within this term are continuous cell lines (e.g., THP-1 cells), primary cell cultures (e.g. monocytes and macrophages isolated from blood), including finite cell lines (e.g., non-transformed cells), cancer cells, and any other cell population maintained in vitro, including tissue and tumors isolated from patients or animals.

As used herein, the term “atheroma” or plural: atheromata” refers to an accumulation and swelling (i.e. -oma) in artery walls that is made up of cells (primarily macrophage cells), and cell debris that contains lipids (cholesterol and fatty acids), calcium and a variable amount of fibrous connective tissue. In the context of heart or artery matters, atheromata are commonly referred to as atheromatous plaques.

As used herein, the term “foam cell” refers to a cell in an atheroma derived from both macrophages and smooth muscle cells that have accumulated low-density lipoproteins, LDLs, by endocytosis. The LDL has crossed the endothelial barrier and has been oxidized by reactive oxygen species produced by the endothelial cells. Foam cells are also known as or form fatty like streaks and typically line the intima media of the vasculature.

As used herein, the term “Low-density lipoprotein” or “LDL” or “bad cholesterol” refers to a type of lipoprotein that transports cholesterol and triglycerides from the liver to peripheral tissues. LDL is one of at least five major groups of lipoproteins; these groups include chylomicrons, very low-density lipoprotein (VLDL), intermediate-density lipoprotein (IDL), low-density lipoprotein, and high-density lipoprotein (HDL) “good cholesterol” or “healthy cholesterol.”

The term “off-target” in reference to side effects refers to unwanted results or even harmful effects to a patient from using system therapeutics, including passive targeting compositions, that bind to or effect cells or pathways that are not involved in the disease undergoing treatment or effect tissue that is not the goal of imaging or undergoing treatment. In other words, off-target is opposite targeted tissue, cells or genes.

The term “nucleotide sequence of interest” refers to any nucleotide sequence (e.g., RNA or DNA), the manipulation of which may be deemed desirable for any reason (e.g., treat disease, confer improved qualities, etc), by one of ordinary skill in the art. Such nucleotide sequences include, but are not limited to, coding sequences, or portions thereof, of structural genes (e.g., reporter genes, selection marker genes, oncogenes, drug resistance genes, growth factors, etc), and non-coding regulatory sequences that do not encode an mRNA or protein product (e.g., promoter sequence, polyadenylation sequence, termination sequence, enhancer sequence, etc).

The term “gene” refers to a nucleic acid (e.g., DNA or RNA) sequence that comprises coding sequences necessary for the production of a polypeptide or precursor (e.g., proinsulin). The polypeptide can be encoded by a full length coding sequence or by any portion of the coding sequence so long as the desired activity or functional properties (e.g., enzymatic activity, ligand binding, signal transduction, etc) of the full-length or fragment are retained. The term also encompasses the coding region of a structural gene and includes sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ untranslated sequences. The sequences that are located 3′ or downstream of the coding region and which are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in involved in up-regulation or down-regulation are often called “activators” and “repressors,” respectively. the process. “Up-regulation” or “activation” refers to regulation that increases the production of gene expression products (i.e., RNA or protein), while “down-regulation” or “repression.”

As used herein, the terms “peptide” or “polypeptide” refer to a chain of amino acids (i.e., two or more amino acids) linked through peptide bonds between the alpha-carboxyl carbon of one amino acid residue and the amide-nitrogen of the next. A “peptide” or “polypeptide” may comprise an entire protein or a portion of protein. “Peptides” and “polypeptides” may be produced by a variety of methods including, but not limited to chemical synthesis, translation from a messenger RNA, expression in a host cell, expression in a cell free translation system, and digestion of another polypeptide.

As used herein the term “protein” is used in its broadest sense to refer to all molecules or molecular assemblies containing two or more amino acids. Such molecules include, but are not limited to, proteins, peptides, enzymes, antibodies, receptors, lipoproteins, and glycoproteins.

As used herein, the term “enzyme” refers to molecules or molecule aggregates that are responsible for catalyzing chemical and biological reactions. Such molecules are typically proteins, such as enzymes, but can also comprise short peptides, RNAs, ribozymes, antibodies, and other molecules.

As used herein, the terms “material” and “materials” refer to, in their broadest sense, any composition of matter.

As used herein, the term “protein of interest” refers to a protein encoded by a nucleic acid of interest.

As used herein, the term “pathogen” refers to disease causing organisms, microorganisms, or agents including, but not limited to, viruses, bacteria, parasites (including, but not limited to, organisms within the phyla Protozoa, Platyhelminthes, Aschelminithes, Acanthocephala, and Arthropoda), fungi, and prions.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., CV Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram-negative bacteria appear red.

As used herein, the term “virus” refers to infectious agents, which with certain exceptions, are not observable by light microscopy, lack independent metabolism, and are able to replicate only within a host cell. The individual particles (i.e., virions) consist of nucleic acid and a protein shell or coat; some virions also have a lipid containing membrane. The term “virus” encompasses all types of viruses, including human, animal, avian, plant, phage, and other viruses.

### DESCRIPTION OF THE FIGURES

FIG. 1 shows exemplary embodiments of targeting molecules (agents) attached to magnetic nanoparticles of the present inventions for use in A) imaging and B) treatment of a patient, while C-E shows an exemplary Scanning Electron Microscope images of C) cholesterol crystals formed in vitro; D) and E) sharp cholesterol crystals in the lumen of the left anterior descending coronary artery of a 35 year old male who died with a heart attack. (The scale bar is 10 μm).

FIG. 2 shows exemplary compositions and structures for glyco-conjugate synthesis A-D synthesis of sugar building blocks A) and B) mannose and C) galactose and D) an alkyne linker (El-Boubbou, et al.; J. Am. Chem. Soc. 2007, 129, 13392-13393; and online supplement; all of which are herein incorporated by reference).

FIG. 3 shows exemplary schematics of construction of A) exemplary coated magnetite iron oxide nanoparticle (NP 1) and MGPN 2-4 B) powder X-ray diffraction (XRD) of NP 1 C) TGA curves for NP 1 (blue), Man-MGNP 3 (brown) and Gal-MGNP 4 (green) and D) FT-IR spectra of bare iron oxide (Fe3O4) NP (brown), NP 1 (blue) and MGNP 3 (black), (El-Boubbou, et al.; J. Am. Chem. Soc. 2007, 129, 13392-13393; and online supplement; all of which are herein incorporated by reference) exemplary methods for use in characterizing nanoparticles of the present inventions.

FIG. 4 shows an exemplary schematic of A) a glyco-conjugate attached to a nanoparticle (MGNP) binding to Con A demonstrating B) fluorescence emission spectra of the supernatants of fluorescein labeled Con A solutions after incubation with NP 1 (5 mg) and various amounts of MGNP 3 followed by magnetic separation, (El-Boubbou, et al.; J. Am. Chem. Soc. 2007, 129, 13392-13393; and online supplement; all of which are herein incorporated by reference).

FIG. 5 shows an exemplary method and application of A) embodiments of glycol-conjugate (targeting ligand) attached magnetic nanoparticles (MGNP-ConA) for use in for detecting B) an E. coli pathogen for imaging and c) for capturing a cell, (El-Boubbou, et al.; J. Am. Chem. Soc. 2007, 129, 13392-13393; and online supplement; all of which are herein incorporated by reference) for use in the present inventions

FIG. 6 shows an exemplary (a) representative fluorescence microscopic images of captured E. coli. The concentration (cells/mL) of bacteria incubated with MGNP 3 (see FIG. 2) is indicated on each image (bd) TEM images of MGNP 3/E. coli complexes, e) demonstration of E. coli strain differentiation by MGNPs 3 and 4. (El-Boubbou, et al.; J. Am. Chem. Soc. 2007, 129, 13392-13393; and online supplement; all of which are herein incorporated by reference).

FIG. 7 shows a demonstration of an exemplary concentration dependent MRI image—where change in image contrast is concentration dependent as demonstrated by T2 weighted MRI images of magnetic NPs a) of the present inventions conjugated mannose (man) compared to b) bare TEOS-NPs (polyvinylpyrrolidone (PVP) as the surfactant and tetraethoxysilane (TEOS)) as shown in c) 0.02 mg NPs incubated with various concentrations of Con A.

FIG. 8 shows an exemplary HA as a) units, b) schematic of crystal structure of CD44, c) a one-pot synthesis of sHA hexasaccharide 5 and d) deprotection of sHA hexasaccharide.

FIG. 9 shows an exemplary schematic of Synthesis of a) HA coated DESPION; b) TEM of HA-DESPION (scale bar is 20 nm), c) HR-MAS 1H-NMR of HA-DESPION, d) PEG linker, e) HA-DESPION binding with macrophage THP cells visualized by Prussian Blue Staining and countered stained with Nuclear Fast Red, f) Media fluoresce intensity measured by FAC Median. TEM of HA-DESPION (the scale bar is 20 nm),

g) Change of hydrodynamic radii of HA-DESPION upon incubation with PBS or 10% FBS containing media; and h) Median fluorescent intensity measured by FACS of vascular endothelial cells EA.hy926 cells due to uptake of HA-DESPION or FITC-DESPION with and without TNF-α treatment.

FIG. 10 shows an exemplary schematic of A) healthy and atherosclerotic plaque, B) schematic showing an exemplary plaque, C) Binding of HA-DESPION with rabbit healthy artery tissues after removal of unbound particles, micrograph of healthy non-atherosclerotic tissue surface after counterstaining; D) Binding of HA-DESPION with rabbit artery tissues after removal of unbound particles, Atherosclerotic tissue surface after counterstaining as in E) Cross section of the atherosclerotic tissue; F) Cross section of the atherosclerotic tissue; and F) MRI image of the atherosclerotic tissue.

FIG. 11A shows exemplary MRI images of rabbit aorta treated with HA-DESPION. a) In vivo MR image of the aorta of a rabbit with early plaques before HA-DESPION injection; b) in vivoimage 10 minutes after HA-DESPION injection; c) ex vivo image following HA-DESPION injection and artery removal with the arrow showing corresponding in vivo slice location; d) in vivo MR image of the aorta of a rabbit with advanced plaques before HA-DESPION injection; e) MR image of the rabbit aorta 10 minutes after HA-DESPION injection; f) MR image of the rabbit aorta 3 hours after HA-DESPION injection; g) in vivo image before HA-DESPION injection; h) in vivo image after HA-DESPION injection; and i) ex vivo image following HA-DESPION injection and artery removal with the arrow showing corresponding in vivo slice location.

FIG. 11B shows exemplary in vivo MR images of the aorta of a rabbit with early plaques treated with HA-DESPION. a) Before HA-DESPION injection; b) 10 minutes after HA-DESPION (1 mg Fe/kg of body weight) injection; c) 10 minutes after injecting DESPION (1 mg Fe/kg) without HA on the surface showing no changes in signal intensity. In vivo MR images of the aorta of a rabbit with advanced plaques d) before HA-DESPION injection; e) 10 minutes, f) 3 hours after HA-DESPION (1 mg Fe/kg of body weight) injection; and g) Quantification of MRI signal intensities (arbitrary unit) of the vessel walls shown in images a)-f) after calibrations against signals at nearby muscle, which were un-affected by NP injections.

FIG. 12 shows an exemplary schematic of preparing a HA nanoparticle of the present inventions A) for linking with a therapeutic, B) lovistatin.

FIG. 13 shows an exemplary schematic of Cyclodextrin (CD) synthesis and linking to a NP of the present inventions.

FIG. 14 shows an exemplary hyaluronic acid (HA, targeting ligand) coated magnetic nanoparticles conjugated to PEG.

FIG. 15 shows an exemplary hyaluronic acid (HA, targeting ligand) coated magnetic nanoparticles conjugated comprising a siTNF-alpha therapeutic a PEG linker for use in targeted drug delivery.

### DESCRIPTION OF THE INVENTION

The present inventions relate to compositions and methods for imaging and treating atherosclerotic diseases, pathogen infections, and tumors by administering actively targeting magnetic nanoparticles. In particular, the present inventions provide new types of targeting ligands attached to magnetic nanoparticles for magnetic resonance imaging. The use of these targeted magnetic nanoparticles is contemplated as a means to treat atherosclerotic diseases, including but not limited to inhibiting and removing atherosclerotic plaques. Further, actively targeting magnetic nanoparticles are contemplated for use with multiple labels for use in nuclear medicine imaging, computed tomography (CT) techniques and other types of imaging for medical and research applications.

Cardiovascular diseases have become the number one cause of death in the world despite the tremendous medical advances over the past decades. Heart disease is one of the leading killers in developed nations. In the United States alone there are approximately 5 million Americans living with heart disease with 550,000 new cases each year. Heart disease in general, includes a number of diseases and factors. Of these, roughly three quarters of the million cardiovascular disease (CVD) deaths each year are due to atherosclerosis, a chronic inflammatory disease of the arterial vessel wall (Crowther, et al., 2005, 436-41; herein incorporated by reference). The prevalence of atherosclerosis is increasing, owing to several factors including an aging population, the escalating pandemics of obesity and sedentary lifestyle and the widespread under-recognition and under-treatment of individuals with risk factors for atherosclerosis.

Currently, diagnosis of atherosclerosis is typically at advanced stages of disease, as detected by the gold standard angiography or cardiac stress testing for overall physiological health, currently the primary diagnostic tools for atherosclerotic plaques. However angiography, merely detects stenosis (an abnormal narrowing in a blood vessel, when arteries are narrowed approximately 50% or more due to plaque and lipid build-up), or by evaluating the effect of stenosis on organ perfusion (Lindsay, et al., Nat. Rev. Drug Disc. 7:517-529, 2008). Neither of these methods yields information about plaque development within the vessel wall. In particular, vulnerable atherosclerotic plaques often do not narrow the arteries by more than 50%, and small plaques are difficult to detect with these conventional diagnostic approaches. While cardiac stress testing indirectly reflects arterial blood flow to the heart during physical exercises this is merely a reflection on a person's overall physical fitness. Early diagnosis of atherosclerosis, i.e. detection of early or small, or growing plaques, would allow for early treatment of the disease, as atherosclerosis is known in some cases to be reversible if treated before the condition worsens.

Newer approaches for visualizing atherosclerotic plaques are published for example, one proposed alternative to angiography is MRI, a technique that can visualize structure and function of the body in high resolution depending upon the contrast agent used. The use of MRI with low or nontoxic contrast agents on humans has minimal side effects when it does not involve the use of radioactive compounds, toxic compounds or invasive procedures (Rodriguez, et al., J. J. Pharm. Sci. 2008, 97, 3637-3665; herein incorporated by reference). MRI relies on the detection of the water proton's relaxation rate in a magnetic field. The changes of the water environment in different body tissues create “background” MR images. MRI has emerged as a powerful non-invasive imaging technique which can provide anatomical information of the plaques (Jaffer, et al., Arterioscler. Thromb. Vasc. Biol. 29:1017-1024 (2009); Jaffer, et al., J. Am. Coll. Cardiol. 47:1328-1338 (2006). To better evaluate the risk of atherosclerosis, the acquisition of molecular and biochemical information becomes crucial (Jaffer, et al., Arterioscler. Thromb. Vasc. Biol. 29:1017-1024, 92009); Jaffer, et al., J. Am. Coll. Cardiol. 47:1328-1338, 2006; Sanz, et al., Nature 451:953-957 (2008); Canet-Soulas, et al., Magn. Reson. Mater. Phy. 20:129-142 (2007); and Briley-Saebo, et al., Probes J. 26:460-479, (2006). This can be accomplished by administering contrast agents, and a popular class of such agents is the paramagnetic Gadolinium (Gd) containing compounds (Jaffer, et al., J. Am. Coll. Cardiol. 47:1328-1338, 2006). To overcome the low relaxivity of Gd, polymeric constructs incorporating multiple Gd ions and ligands have been utilized to target several components of plaques (Jaffer, et al., J. Am. Coll. Cardiol. 47:1328-1338, 2006; Sanz, et al., Nature 451:953-957, 2008; Canet-Soulas, et al., Magn. Reson. Mater. Phy. 20:129-142, 2007; Briley-Saebo, et al., Probes J. 26:460-479, 2006) which include macrophage scavenger receptor (Lipinski, et al., Proc. Nat. Acad. Sci., USA 961-966 104 2007), fibrin in thrombus (Nair, et al., Chem. Int. Ed. 4918-4921 47 2000) and integrin (Winter, et al., JACC: Cardiovasc. Imaging 624-634 1 2008; Winter, et al., Arterioscler. Thromb. Vasc. Biol. 2103-2109 26 2006). Besides imaging, an elegant theranostic approach has been developed using the alpha, beta₃ integrin targeting paramagnetic Gd constructs to deliver an anti-angiogenic drug fumagallin, which led to reduced angiogenesis in an atherosclerotic rabbit model (Winter, et al., JACC: Cardiovasc. Imaging 624-634 1 2008; Winter, et al., Arterioscler. Thromb. Vasc. Biol. 2103-2109 26 2006). Several of these novel assemblies are expected to enter clinical trials in the next few years. A complication in using Gd, however, is the recent association of Gd usage with patients developing nephrogenic systemic fibrosis, an untreatable fatal disease (Bhave., et al., Systemic Fibrosis J. Urology 830-835 180 2008). Examples of alternative agents for MR molecular imaging as described in Neuwelt, et al., Kidney Int. 465-474 75 2000). Numerous studies are published, particularly using rabbits, pigs, and nonhuman primates, showing various images of plaque components as “background” images, i.e. images obtained without the use of a specific contrast agent (for examples, (Fayad, et al., Circulation 1998, 98, 1541-1547; Helft, et al., J Am Coll Cardiol, 2001; 37:1149-1154; herein incorporated by reference).

More recently, general or “passive” contrast agents are being used for MRI imaging of plaques. Specifically, “passive” contrast agents that are phagocytosed by macrophages were used for imaging plaques. In particular, ultra small super paramagnetic particles of iron oxides (USPIOs) (Sinerem™, Guerbet) are phagocytosed by cells of the mononuclear phagocytic system in rabbits, for one example, Ruehm, et al., Circulation 2001; 103; 415-422; herein incorporated by reference).

The superparamagnetic iron oxide nanoparticles (SPIONs) or SPIO nanoparticles (see, Thorek, et al., Ann. Biomed. Eng. 34:23-38 2006; Corot, et al., Adv. Drug Del. Rev. 1471-1504 58 2006; Wang, et al., Eur. Radiol. 11:2319-2331 2000) were used in MRI applications. With their large relaxivity and high biocompatibility, SPIONs were excellent MRI probes under certain conditions. The primary approach for atherosclerosis detection using SPIONs was to take advantage of the high capacity of macrophages to uptake particles, albeit in a non-specific manner (Sigovan, et al., Animal Model Radiology 401-409 252 2009; Morris, et al., Arterioscler. Thromb. Vasc. Biol. 265-271 28 2008; Durand, et al., J. Vasc. Res. 119-128 44 2007; Ruehm, et al., Circulation 103:415-422 (2000). Due to the large number of macrophages present in the plaque, contrast can be obtained allowing plaque imaging and monitoring.

This approach was tested in mouse subjects for imaging of atherosclerosis development, Trogan, et al., Arterioscler., Thromb., Vasc. Biol. 2004, 24, (9):1714-1719; Hockings, et al., Circulation 2002, 106(13):1716-1721; herein incorporated by reference), in coronary arteries and heart valves (Ruff, et al., J. Magn. Reson. 2000, 146 (2):290-296; herein incorporated by reference) and atherosclerotic lesions (Fayad, et al., Circulation 1998, 98(15):1541-7; herein incorporated by reference) and have produced excellent data in terms of resolution and characterization of the targeted organ. Due to the large number of macrophages present in the plaque, contrast can be obtained allowing plaque imaging and monitoring. Further, these nanoparticles used for MR imaging are “passive” nanoparticles not “active” or “smart” nanoparticles of the present inventions such that the passive particles are merely nonspecifically pahgocytosed by any cell, such that these USPIOs lacking a targeting ligand would not be of use for specific diagnostic imaging nor for specific targeting of therapeutic molecules. However this approach has limitations when using a larger type of animal. Moreover, due to the non-targeted nature of the NP utilized in this approach, high concentrations of NP (56 mg Fe/Kg body weight in rabbit model) were required (Sigovan, et al., Animal Model Radiology 401-409 252 2009; Morris, et al., Arterioscler. Thromb. Vasc. Biol. 265-271 28 2008; Durand, et al., J. Vasc. Res. 119-128 44 2007; Ruehm, et al., Circulation 103:415-422 (2000)). As a result, imaging had to be performed at least one day with the optimum window of 4 to 5 days after injection to allow sufficient clearance of the NP from the background and NP uptake by macrophages. To improve the plaque selectivity, various targeting ligands have been immobilized onto SPIONs, which include phage display-derived peptide binders and mAb for VCAM (Radermacher, et al., Invest. Radiol. 44:398-404, 2009; McAteer, et al., Arterioscler. Thromb. Vasc. Biol. 28:77-83, 2008; Smith, et al., Biomed. Microdevices 9:719-727 (2007)) annexin V for apoptotic cells Sosnovik, et al., Magnetic Resonance Imaging of Cardiomyocyte Apoptosis with a Novel Magneto-Optical Nanoparticle Magn. Reson. Med. 718-724 54 2005; Nahrendorf, et al., Imaging Identifies Inflammatory Activation of Cells in Atherosclerosis Circulation 1504-1511 114 2006) and mAb and small molecule ligand for selectin (Kelly, et al., Circ. Res. 327-336 96 2005; McAteer, et al., Arterioscler. Thromb. Vasc. Biol. 28:77-83, 2008, Radermacher, et al., Spectroscopy Invest. Radiol. 44:398-404, 2009; Smith, et al., Biomed. Microdevices 9:719-727, 2007; Sosnovik, et al., Magn. Reson. Med. 54:718-724, 2005; Nahrendorf, et al., Circulation 114:1504-1511 2006; Kang, et al., Bioconjugate Chem. 13:122-127 2002. Some probes have been employed to evaluate plaques in vivo via MRI. However, in many of these studies, high concentrations of NPs (56 mg Fe/Kg body weight in rabbit model) and/or prolonged delay after injection were still necessary. Furthermore, to accurately evaluate the risks of acute vascular events, multi-parameter analysis is highly desired. This necessitates the continual search for new markers and probes for molecular imaging.

The quality of MRI images for a specific organ, cell, or tissue is sometimes greatly enhanced by delivering a paramagnetic contrast agent to the region of interest, with the most popular contrast agents being the paramagnetic metal Gadolinium (Gd) compounds such as GdDTPA. To further improve detection, Gd was attached onto ligands to target several different components of plaques, such as LDL, fibrin, and MMP (Ponta, et al., Nature Rev. Mol. Cell. Biol. 2003, 4, 33-45; Briley-Saebo, et al., J. Mag. Res. Imag. 2007, 26, 460-479; herein incorporated by reference). However, the recent association of Gd exposure with the onset of nephrogenic systemic fibrosis, an untreatable disease in humans, has generated strong interest in using alternative safer contrast agents for MRI molecular imaging.

Therefore, the present inventions provide numerous exemplary contemplated embodiments of compositions and methods comprising safer alternatives to Gd nanoparticles, such as superparamagnetic iron oxide nanoparticles (SPION)s which are used as T2/T2* based contrast agents for MRI due to their capabilities to reduce transverse magnetization of protons. Further the inventors contemplate such as magnetic nanoparticles (NPs) coated with targeting ligands for actively targeting molecules and cells found associated with and in atherosclerotic plaques.

Advantages of using targeted magnetic nanoparticles (NPs) of the present inventions, in part as an alternative to associated toxic effects of Gd nanoparticle exposure, are that in some embodiments, the inventors contemplate nanoparticles such as superparamagnetic iron oxide nanoparticles (SPIONs) which are being used in medicine in a variety of fields such as in biosensors and MRI (Gupta, et al., Biomaterials 2005, 26, 3995-4021; Laurent, et al., Chem. Rev. 2008, 108, 2064-2110; herein incorporated by reference).

In a preferred embodiment, targeting nanoparticles of the present inventions are contemplated for identifying and treating plaques at any stage, such as pre-plaques, early plaques, and vulnerable plaques.

Local inflammation in involved with the earliest onset of atherosclerosis disease and plays a critical role in every stage of atherogenesis, such as in plaque formation. One of the initial steps in forming atherosclerotic lesions, and inducing inflammation, begins when circulating oxidized low-density lipoprotein (ox-LDL) contacts an artery wall or circulating LDL becomes oxidized by the endothelium causing endothelial damage, which induces the expression of adhesion molecules on the endothelial lining of the arterial blood vessel (lumen). Thus in one embodiment, the inventors contemplate targeting magnetic nanoparticles for reducing or altering the interaction of ox-LDL or LDL with artery lumen for reducing damage to an artery wall, i.e. reducing damage that would lead to plaque formation. In other words, the inventors contemplate the advantage of detecting damaged arterial cell wall associated with plaque formation, i.e. pre-plaque areas.

One of the physiological responses to ox-LDL arterial injury, for example, in humans, is white blood cell and leukocyte recruitment, such as macrophages and T-lymphocytes, in response to the over-expression of adhesion molecules and interaction with these adhesion molecules at the site of pre-atherosclerotic lesion, leading to formation of a plaque. Therefore, in one embodiment, the inventors contemplate targeting ligands for target adhesion molecules, such as CD44, Vascular Cell Adhesion Molecule-1 (VCAM-1), Inter-Cellular Adhesion Molecule-1 (ICAM-1), etc., located on the endothelial cells lining the interior of artery wall for imaging and for therapeutic treatment for slowing or stopping the formation of an early plaque (i.e. altering the damage endothelial cell such that the formation of a plaque is inhibited). Pre and early lesions are associated with macrophages and smooth muscle cells of the arterial wall, which ingest (endoyctose) ox-LDL to become foam cells. These foam cells can grow and undergo premature cell death (necrosis and apoptosis), releasing a greater amount of concentrated cholesterol (and other lipids) from the ingested LDL into the artery wall. This lipid injection into the wall attracts more white blood cells to the plaques, perpetuating the inflammatory cycle. The white blood cells associated with the plaques express adhesion molecules similar to that of the endothelium as described above. Therefore, the inventors contemplate targeting ligands for white blood cell activation molecules attached to magnetic nanoparticles of the present inventions for imaging plaques and inhibiting the growth of these plaques.

Several types of physiological events associated with the formation and growth of plaques contain a variety of molecules contemplated as targets for plaque imaging and treatment. For one example, apoptotic white blood cells unleash enzymes such as matrix metalloprotease-2 and -9 (MMP-2 and MMP-9, respectively) (Beaudeux, et al., J. Clin. Chem. Lab. Med. 2004, 42, 121-131; herein incorporated by reference). Although MMP involvement is beneficial for remodeling the extracellular matrix in order to accommodate the increasing cell mass during normal growth, MMPs are considered detrimental to the health of the atherosclerotic patient. For example, higher activities of MMPs associated with plaques cause thinning of a fibrous cap that typically separates the plaques from the blood plasma of the vessel. Small blood vessels can initiate and grow into the plaques (angiogenesis), which also contribute to plaque growth and instability, e.g. vunerable plaques. Eventually, the unstable plaques, rich in cholesterol and macrophages, rupture and cause heart attack and stroke.

Thus, the capability to detect early plaque formation as well as to differentiate various developmental stages of plaques, currently lacking, is highly desirable (Sanz, et al., Nature 2008, 451, 953-957; herein incorporated by reference).

In general, a plaque is characterized by the presence of prominent lipid core, a thin fibrous cap and high density of inflammatory cells. As the disease progresses, the plaque continues to grow leading to further thinning of the fibrous cap that can eventually rupture causing a sudden cardiac death.

Vulnerable plaques are characterized by extensive macrophage infiltration, thin fibrous cap and large quantities of cholesterol crystal, a wide range of agents, such as anti-inflammatory cortisone steroid, nitric oxide (NO), siRNA, cholesterol lowering statins, angiogenesis inhibitors, and MMP inhibitors are contemplated to potentially benefit atherosclerosis treatment (Spratt, et al., 2004, 90, 1392-1394; herein incorporated by reference). However, many of these agents cannot be used directly as systemic application of compounds, for example, cortisone steroid, NO, etc., at potentially therapeutic levels is likely to cause harmful “off-target” side effects when used alone or in combination with passive agents.

Currently the primary diagnostic tools for atherosclerosis are cardiac stress testing or angiography that are used for detecting arteries with narrowing typically at least 50%. This renders stress testing and angiography as unreliable for detecting venerable plaques since vulnerable plaques typically narrow arteries by much less that 50%. There fore MRI is being used for direct imaging of atherosclerotic plaques with nonspecific or passive types of contrast agents for imaging these plaques. Medical and research evidence indicates that activated macrophages initiate, maintain, and aggravate the various step of plaque formation that lead to plaque rupture and blood vessel blockage.

Target extracellular matrix, cholesterol, and low-density lipoprotein (LDL) magnetic nanoparticles with super-paramagnetic properties for enhancing contrast of the plague compared to the surrounding tissue allowing clear plaque detection by MRI. These particles will be tested in mice and rabbits, and ex vivo staining of atherosclerotic tissues. Contemplate in one embodiment, detecting early and unstable plaques. Contemplate in one embodiment, site-selectivity of delivering therapeutic agents. Image based therapeutics will provide conclusive evident that the drug is reaching the desired site and the molecular effect can be easily monitored

Further, contemplate using several types of imaging (contrast) agents, such as PET. CT etc., each separately in combination with a unique targeting ligand for a different part of plaque components for enabling the monitoring and differentiation of the molecular and cellular events occurring at the atherosclerotic plaque, allowing detailed characterization of the plaques. Even further, the inventors contemplate the use of combining targeted drub delivery and molecular imaging with MRI for providing an area of cardiology research not available at this time. For example, research and treatment methods comprising nanoparticles of the present inventions may be used for developing innovative personalized therapy, for identifying specific ligand targets and therapies for reducing cardiac events, and the like.

Therefore, the inventors contemplate these agents as therapeutics attached to targeting magnetic nanoparticles of the present inventions. Advantages of using targeted delivery compositions to overcome current limitations such as “off target” side effects and for providing superior methods of treatments is contemplated by using a specific targeting ligand in combination with a nanoparticle to achieve high local concentration and longer retention time of therapeutic agent at the plaque site without resorting to systemic administration of high dosages.

Further contemplated is the advantage of extending patient life (i.e. reducing cardiac events) by using magnetic nanoparticles of the present inventions comprising targeting ligands for target molecules expressed on cells associated with plaques in both imaging and therapeutic applications. Specifically, the inventors contemplate that by using specific targeting molecules instead of passive molecules, the clarity and thus the identification and interpretation of the images of plaques would be significantly increased.

### I. Target Analyte and Ligand Molecules.

A. Targeting Molecules.

CD44 is a cell surface receptor with its principle ligand being hyaluronic acid (HA) (Ponta, et al., Nature Rev. Mol. Cell. Biol. 2003, 4, 33-45; herein incorporated by reference). The interaction between CD44 and HA was used for targeted delivery of agents by coated polyplexes to corneal epithelial (Hornof, et al., J. Gene Med. 2008, 10, 70-80, herein incorporated by reference) and cancer cells (Jaracz, et al., Med. Chem. 2005, 13, 5043-5054 and references cited therein; all of which are herein incorporated by reference). HA/CD44 binding was shown to facilitate CD44 mediated uptake, decrease cytotoxicity of the delivered agents and reducing side effects. HA/CD44 interactions have been implicated in a variety of physiological and pathological processes including cancer metastasis (Trochon, et al., Int. J. Cancer 1996, 66, 664-668; herein incorporated by reference angiogenesis (Trochon, et al., Int. J. Cancer 1996, 66, 664-668; herein incorporated by reference) inflammation (DeGrendele, et al., Science 1997, 278, 672-675; herein incorporated by reference) and arthritis (Naor, et al., Arthritis Res. Ther. 2003, 5, 105-115; herein incorporated by reference). In relation to atherosclerosis, inflammatory signals transform CD44 from an inactive, nonbinding form to its high affinity form through sufation (Maiti, et al., Science 1998, 282, 941-943; herein incorporated by reference which promotes the adhesion of activated macrophages to endothelium and smooth muscle cells via multiple mechanisms (Cuff, et al., J. Clin. Invest. 2001, 108, 1031-1040; herein incorporated by reference).

As a marker for inflammation, activated CD44 exists on both activated macrophages and vascular endothelial cells throughout the plaque development stages (Jain, et al., J. Clin. Invest. 1996, 97, 596-603; McKee, et al., J. Clin. Invest. 1996, 98, 2403-2413; herein incorporated by reference).

Hyaluronic acid (HA), a long chain glycosaminoglycan, is a critical molecular effector of vascular cell phenotype. CD44, the principal cellular receptor for HA, is one of the crucial glycoproteins that is upregulated after vascular injury and is a prominent component in atherosclerotic and restenotic lesions. Cell-matrix interactions mediated by the CD44/hyaluronic acid receptor-ligand pair are involved in the recruitment of inflammatory cells to the vessel wall and activation of vascular cells. CD44 expression and the accumulation of its ligand HA both increase in atherosclerosis and restenosis. Thus, attention must be given to the targeting of HA and CD44 as strategies to block the development of both diseases that claim more lives in the western world than any other diseases. Moreover, for determining effects of multivalency on HA/CD44 interactions polyvalent sHA constructs, such as sHA-coated magnetic nanoparticles (MNPs) as well as fluorescent sHA MNPs are contemplated. Moreover, the use of sHA immobilized MNPs will offer a unique opportunity to probe atherosclerotic plaques by magnetic resonance imaging (MRI). In vivo cellular MR tracking with sHA coated nanoparticle probes has the potential for determining the fate of the cells and evaluating cell-based therapies. The detailed understanding of the structure-activity relationship and availability of these sHA compounds will provide valuable tools for characterizing sHA interaction with its cellular receptor CD44 and broaden our understanding of the role CD44 plays in the reaction of vascular smooth muscle cells to arterial wall injury.

B. CD44 in Atherosclerosis and Methods for CD44 Targeting in Atherosclerotic Plaques.

In recent years, much evidence has accumulated supporting that inflammation plays a key role in the pathogenesis of atherosclerotic diseases. Atherosclerosis begins when oxidized LDL comes in contact and damages an artery wall causing upregulation of adhesion molecules on the endothelial cells lining the interior of artery wall. Human bodies respond to this injury by recruiting inflammatory cells such as macrophages to the lesion sites through cooperative binding of multiple endothelial cell surface adhesion molecules including VCAM, integrin, selectin and CD44. This process is essential for plaque development.

CD44 is a cell surface receptor expressed on three major cell types present in the atherosclerotic plaques, i.e., vascular endothelial cells, macrophages and smooth muscle cells. Multiple studies have suggested that CD44 promotes atherosclerosis by mediating inflammatory cell recruitment and vascular cell activation. In an atherosclerotic mouse model the ApoE knockout mice, CD44 expression was found to vary in vascular vessels and was highest at lesion prone sites. This was corroborated in human studies, as CD44 is present in rupture-prone macrophage-rich regions of human atherosclerotic plaques at a level over 10 folds higher than that in healthy vascular tissues. CD44 was also found over-expressed in atherosclerotic rabbits. The elevated CD44 expression correlated with 10 fold enhancement of the secretion of proinflammatory cytokines such as IL1beta and IL-6 from endothelial cells and macrophages and these cytokines in turn augmented CD44 expression. Such a positive feedback loop can exacerbate atherosclerosis development leading to plaque instability. The knockout of CD44 in ApoE knockout mice led to 50˜70% reduction in aortic lesions as well as 90% fewer macrophages present in the lesions. These evidences strongly suggest the critical functions of CD44 in plaque initiation and development. However, CD44 was not investigated as a target for molecular imaging of atherosclerotic plaques.

The principle ligand of CD44 is HA, a naturally existing polysaccharide. Although CD44 can also be found on non-plaque residing cells, the interactions between HA and CD44 are subject to tight regulations such that the receptor is normally maintained in a quiescent state showing little appreciable HA binding. For example, although freshly isolated peripheral blood monocytes and lymphocytes express CD44, they do not bind soluble HA. During inflammation, however, the pro-inflammatory cytokine TNF-alpha induces sulfation and subsequent conformational changes of CD44, which transforms it into a form with much greater HA affinities. The non-covalent interaction between endothelial cell surface CD44 and HA is sufficiently strong to provide resistance to shear under physiologic conditions and thereby help mediate lymphocyte rolling and facilitate macrophage extravasation into the sites of plaque formation. The over-expression of CD44 in plaques coupled with the enhanced affinity of CD44 with HA during inflammation renders it attractive to utilize HA/CD44 interactions for molecular imaging of plaques.

In addition to HA, CD44 can bind with other extracellular matrix components such as fibronectin and collagen. Digestion of fibronectin and subsequent fractionation studies have identified that a KNNQKSEPLIGRKKT oligo-peptide sequence of fibronectin is responsible for 65.5% of the binding of CD44 to intact fibronectin. In a similar fashion, a collagen fragment of GVKGDKGNPGWPGAP was found to bind with CD44 on melanoma cells but not by the collagen-binding integrins or melanoma-associated proteoglycan. In some embodiments, it is contemplated that these peptides can be used as ligands for CD44 targeting. Besides these peptides, anti-CD44 monoclonal antibodies (mAb) are another option, as several anti-CD44 mAbs have been used to target CD44 on hematopoietic stem cells in vivo.

In order to attach these peptides, magnetic NPs bearing amines on the surface will be first functionalized with a hetero-bifunctional linker SMCC to introduce maleimide groups onto the NPs. The peptides will be assembled through automated synthesis and an additional cysteine will be added to the N-terminal of the peptides. The thiol group of the peptide will chemoselectively ligate with the maleimide bearing NPs through a Michael reaction to introduce the peptides onto the nanoparticles. The amount of peptides attached will be determined through TGA and HRMAS-NMR analysis of the particles. In addition to HA and CD44 recognizing peptides, the attachment of an anti-CD44 mAbs onto magnetic NPs is contemplated. There are a variety of anti-CD44 mAbs commercially available against human (MEM85), rabbit (W4/86) and mouse CD44 (IM7.8.1). These mAbs will be immobilized onto magnetic NPs through SMCC. The colloidal stability, biocompatibility and CD44 binding of these anti-CD44 NPs will be evaluated in a similar manner as the HA-NP.

To further minimize the non-specific uptake, a new synthetic type of HA-NPs was developed without dextran coating. Thermal decomposition of Fe(acac)₃ in the presence of oleic acid produced highly homogenous magnetic nanoparticles with an average core size of 6 nm. Mixing these particles with HA under a biphasic system of water and toluene exchanged the oleic acid ligands with HA, rendering them highly water soluble. These particles are colloidal stable with average hydrodynamic radii of 90 nm and a R₂ value of 230 mM⁻¹ s⁻¹ at 3 T. It has been shown that polyethylene glycol (PEG) can significantly reduce non-specific cellular uptake of NPs by cells including macrophages. To introduce PEG, the oleic acid coated magnetic NPs can be functionalized with amines through ligand exchange with 3-aminopropyl triethoxysilane. PEG and HA will then be co-immobilized as surface ligands through amide bond formation. The effects of surface ligand density, molecular weight of HA immobilized, PEG to HA ratio on HA-NP/CD44 interactions will be explored. The optimal construct with minimum non-specific uptake while maintaining HA-CD44 recognition will be identified through ELISA and flow cytometry analysis of cellular uptake.

In an atherosclerotic mouse model, the knockout of CD44 led to a 50˜70% reduction in aortic lesions (Cuff, et al., J. Clin. Invest. 2001, 108, 1031-1040; herein incorporated by reference). Furthermore, HA overexpression at human atherosclerotic lesions is found to promote atherosclerosis development through binding with CD44 (Chai, et al., Circ. Res. 2005, 96, 583-591; herein incorporated by reference). These findings highlight the important role of CD44/HA interaction plays in atherosclerosis. Targeting CD44 may allow us to monitor inflammation throughout plaque development leading to early detection of plaques. This is crucial for reducing plaque progression and development. The unique interaction between CD44 and HA was exploited for targeted delivery of agents to epithelial and cancer cells (Jaracz, et al., Med. Chem. 2005, 13, 5043-5054 and references cited therein; all of which are herein incorporated by reference). HA/CD44 binding was shown to facilitate CD44 mediated uptake, decrease cytotoxicity of the delivered agents and reduce side effects. However, CD44 has not been explored for atherosclerotic plaque binding.

2. Targeting the CD44 Cell Adhesion Molecules in Atherosclerotic Plaques.

One of the key understandings of the atherosclerotic plaque reveals that HA is present in regions of atherosclerotic lesion. Hyaluronan interacts with cells through its principle receptor CD44, typically present on the endothelial cells as well as active macrophages (Protasiewicz, et al., Pol. Merkuriusz Lek. 2005, 19, (112), 559-562; herein incorporated by reference).

This ligand-receptor pair was implicated in a variety of disease progression stages like metastasis of cancer tumors (Trochon, et al., Int. J. Cancer 1996, 66, (5), 664-668; herein incorporated by reference) angiogenesis (Trochon, et al., Int. J. Cancer 1996, 66, (5), 664-668; herein incorporated by reference) inflammation (Termeer, et al., J. Immunol. 2000, 165, (4), 1863-1870; herein incorporated by reference) and arthritis (Naor, et al., Arthritis Res. Ther. 2003, 5, (3), 105-115; herein incorporated by reference) due to its ability to influence cell behavior. In case of atherosclerosis, CD44 anchors HA on the surface of endothelial cells and thus facilitates recruitment of leukocytes to the site of lesion. Leukocytes once differentiated into macrophages, also express CD44 and enhance the inflammatory cascade. The unique interaction between CD44 and HA can be exploited to target the atherosclerotic plaque. Further, in one embodiment, it is contemplated to disguise the nanoparticles carrying siRNA with HA oligomers to achieve site-specific delivery to the plaque. Hyaluronan and Hyaluronic Acid Oligosaccharides Hyaluronic acid (HA) also known as Hyaluronan is a naturally occurring biopolymer predominantly found in connective, epithelial and neutral tissue (Knudson, et al., Matrix Biol. 2002, 21, (1), 15-23; herein incorporated by reference). It also a key component of extracellular matrix, playing a vital role in number of biological processes like cell proliferation, cell adhesion, cell migration, and inflammation (Lapcik, et al., Chem. Rev. 1998, 98, (8), 2663-2683; herein incorporated by reference).

Structurally, HA belongs to the family of glycosaminoglycans and is made of repeating units consisting of D-glucuronic acid-1,3-D-N-acetyl glucosamine disaccharide connected via β-1,4 glycosidic linkages. Although the molecular weight of the native HA polymer is typically in millions, it can be transformed in vivo into oligosaccharide fragments (sHA) (Knudson, et al., Matrix Biol. 2002, 21, (1), 15-23; herein incorporated by reference).

Recent discoveries have revealed that the sHA have unique biological properties distinct from their bigger counterparts (Ghatak, et al., J. Biol. Chem. 2002, 277, (41), 38013-38020; Misra, et al., J. Biol. Chem. 2003, 278, (28), 25285-25288; herein incorporated by reference). The studies performed to estimate the binding properties of HA and CD44 indicate that the minimum size of oligosaccharide required to replace native high molecular HA from the cell surface is about 6 saccharide units (Teriete, et al., Mol. Cell. 2004, 13, (4), 483-496; herein incorporated by reference).

In terms of availability, sHA was obtained by enzymatic degradation of HA polymers obtained from animal sources or fermentation (Mahoney, et al., Glycobiology 2001, 11, (12), 1025-1033; herein incorporated by reference). Although this approach leads to a quick synthesis of naturally occurring sHA, it is not the rational approach for the synthesis of modified sHA analogs. The chemically modified sHA oligosaccharides are currently being synthesized by the inventors will be screened to obtain HA analogs with enhanced binding efficacy to CD44.

In particular, the present inventions provide a hyaluronic acid (HA) magnetic nanoparticle (targeted nanoprobe) for molecular imaging of atherosclerotic plaques by MRI. These targeted nanoprobes are contemplated for use as an efficient tool for the early detection of atherosclerosis, for example, by targeting the HA receptor, CD44, a molecule present at the earliest stages of atherosclerotic plaque formation, by targeting cholesterol crystal using cyclodextrin magnetic nanoparticles, and the like.

In one embodiment, the inventors contemplate diagnostic clinical use of MRI molecular imaging techniques for showing HA (or other targeting ligand, such as cyclodextrin) coated magnetic nanoparticles within a patient for measuring plaque status. In one preferred embodiment, the plaques are imaged before clinical symptoms are present. In another preferred embodiment, the plaques are imaged in early stages. In another preferred embodiment, the inflamed arterial cell walls prior to plaque formation are imaged.

C) Cholesterol and Atherosclerotic Plaques.

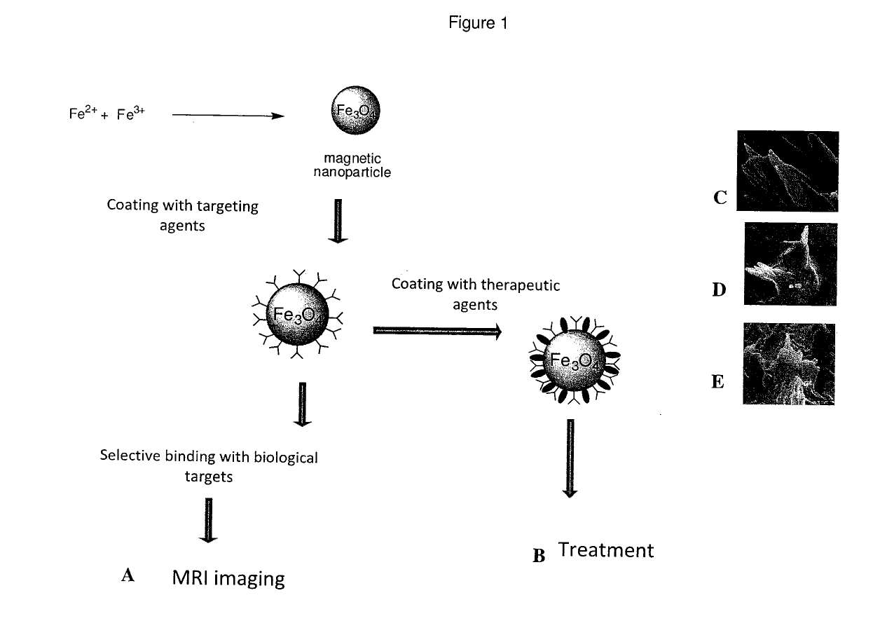

Cholesterol has been intimately linked with atherosclerosis development, as high level of cholesterol is the most commonly used prognosis for cardiovascular diseases. Cholesterol crystal is present in large quantities in vulnerable plaques as a result of foam cell death, which recruits more macrophages and perpetuates the vicious cycle of inflammation. (Abela, et al., American Journal of Cardiology 2009 (103(7): 959-968, all of which is herein incorporated by reference). In addition to its role in inflammation recent discoveries showed that cholesterol expands significantly when it crystallizes (Vedre, et al., Atherosclerosis 2009, 203: 89-96, and references cited therein; all of which are herein incorporated by reference). The volume expansion in the confined space of an atheromatous plaque can exert mechanical outward pressure on the plaques and lead to plaque eruption. Furthermore, the cholesterol microcrystals formed have sharp edges, which can puncture the fibrous lining, weakening the cap and destabilizing the plaques (FIG. 1 (C-E). This novel finding was subsequently supported by extensive microscopic evaluation of human plaques from patients who died with acute cardiac events or had strokes (Vedre, et al., Atherosclerosis 2009, 203, 89-96 and references cited therein; all of which are herein incorporated by reference; Abela, et al., American Journal of Cardiology 1 Apr. 2009 (Vol. 103, Issue 7, Pages 959-968, published online 9 Feb. 2009, all of which is herein incorporated by reference). The cholesterol crystals piercing through the plaques can also break off and shower into downstream organs, which can obstruct blood vessels and cause organ failure (cholesterol crystal embolism).

Therefore, the presence of cholesterol crystals on the surface and inside the plaques may be an important marker for plaque instability and rupture. In vivo detection of cholesterol crystals in atherosclerotic plaques is contemplated to provide an early screening tool for patients at high risk for heart attacks and strokes.

### II. Nanoparticles for Imaging and Therapeutic Treatment.

Superparamagnetic iron oxide nanoparticles with appropriate surface chemistry can be used as contrast agents in MRI due to their ability to enhance proton relaxation. The nanoparticles should exhibit highly colloidal properties, low toxicity and no discrete magnetic properties to be effective delivery vehicles and have excellent MRI applicability (Sun, et al., Adv. Drug Delivery Rev. 2008, 60, (11), 1252-1265; herein incorporated by reference).

In general, DDT-INV nanoparticle (NP) based platform technology is contemplated for detecting plaques, such as vulnerable plaques, and to monitor plaque development and deliver therapeutic agents. Magnetic NPs with high affinity ligand immobilized on the external surfaces selectively binds with atherosclerotic plaques allowing their imaging by Magnetic Resonance Imaging (MRI). Furthermore, these NPs can be decorated with therapeutic agents for targeted therapy of atherosclerotic plaques.

A. Nanoparticles for Magnetic Resonance Imaging (MRI).

Nanoparticles contemplated for use in the present inventions include but are not limited to those described herein. Any supramagnetic nanoparticle for providing an clear MRI image and non-toxic to an animal are contemplate for use in the present inventions. For example, anionic clay, layered metal hydroxide nanoparticle, a high contrast enhancement, with low toxicity and flexible surface chemistry, was contemplated for use as an efficient vehicle for drug delivery and cancer cell targeting (Sun, et al., Adv. Drug Del. Rev. 2008, 60, 1252-1265; herein incorporated by reference).

The magnetic nanoparticles of the present invention generally comprise a solid support magnetic core particle in the nanometer size range. The core particle employed to construct the ligand conjugated magnetic nanoparticles of the present invention are preferably small particles (e.g. nanometer range) that effectively serve as a solid support or solid phase for conjugation to a plurality of ligand molecules and used in conjunction with mass spectrometric methods. Even though particles can be of any size, the preferred size is 0.1-500 nanometers, preferably 1-150 nanometers, more preferably 5-15 nanometers, and most preferably about 9.0 nanometers. The particles may be uniform (e.g., being about the same size) or of variable size. Particles may be any shape (e.g. spherical or rod shaped), but are preferably made of regularly shaped material (e.g. spherical). The particles of the present invention are preferably composed of material that exhibits superparamagnetic properties.

Magnetic nanoparticles may be composed of any type of material that exhibits magnetic properties and do not aggregate in water or in an animal. For example, the nanoparticles useful in the present invention may be composed of a metal, such as iron, nickel, cobalt, and alloys of these metals. In certain embodiments, the magnetic nanoparticles are composed of ceramic material. Preferably, the magnetic nanoparticles are composed of material exhibiting superparamagnetic properties (e.g. particles that can be magnetized with an external magnetic field but dispersed simultaneously once the magnet is removed).

B. Other Types of Imaging

The inventors further contemplate attaching multiple imaging agents to the nanoparticles of the present inventions for applications including multimodal imaging, fluorescence, and PET.

### III. Representative Therapeutic Applications.

In additional contemplated embodiments include targeting ligand (active) nanoparticles of the present inventions comprising a therapeutic for increasing the health and lifespan of a patient.

Early onset of atherosclerosis is marked by the inflammatory cascade where the endothelial cells overexpress a variety of adhesion molecules including vascular cell adhesion molecule-1 (VCAM-1), intercellular adhesion molecule (ICAM-1), selectins and CD44 (Crowther, et al., Hematology Am Soc Hematol Educ Program 2005, 436-41; herein incorporated by reference). This leads to recruitment of leukocytes that involves hyaluronan anchored to the surface of endothelial cells via CD44. Activation and modulation of these cell adhesion molecules is orchestrated by proinflammatory cytokines, typically TNF-α, interleukins, interferon (IFN)-γ, and colony stimulating factors (Kleemann, et al., Cardiovasc. Res. 2008, 79, (3), 360-376; herein incorporated by reference).

Upregulation of TNF-α has been shown to be a key factor in ICAM-1 and VCAM-1 expression as well as CD44 activation in the atherosclerotic lesion (Elkind, et al., Stroke 2002, 33, (1), 31-38; herein incorporated by reference).

Scientific research has shown that inhibition of TNF-α leads to minimization of expression of the adhesion molecules and a consequent decrease in macrophage recruitment and hence has become an important therapeutic target (Branen, et al., Arterioscler., Thromb., Vasc. Biol. 2004, 24, (11), 2137-2142; herein incorporated by reference). However, passive or nonselective (i.e. systemic) anti-TNF-alpha treatment is associated with unwanted harmful side effects including harmful medical complications. Further, it is significantly difficult to selectively suppress the TNF-α activity in the arterial plaque, as TNF-α is a common proinflammatory cytokine that participates in a variety of immunological responses. For example, a systemic suppression of TNF-α might prevent an inflammatory response at the sight of a bacterial infection, leading to deadly side effects. To address the lack of targeted delivery agents the inventors contemplate the use of active nanoparticles with high specificity (a targeting ligand) for a molecule associated with the atherosclerotic plaque, further showing low toxicity to the patient by using higher loading volumes than can be safely used in nonspecific anti-TNF-alpha therapy. Specifically, the inventors contemplate a targeting nanoparticle of the present inventions for use as a therapeutic.

Thus in one embodiment, the inventors contemplate design, synthesis and biological studies for providing a unique delivery vehicle for site specific knockdown of TNF-α using the TNF-α siRNA. The inventors do not intend to limit a therapeutic active nanoparticle to patients with atherosclerosis; indeed the inventors contemplate a variety of targeting ligands specific for other types of diseases, such as Alzheimer's, cancer and pathogen infections.

Compositions and methods for providing small interfering RNA (siRNA) or microRNA (miRNA), otherwise known as “short interfering RNA” or “silencing RNA” mediated silencing of proinflammatory cytokines using RNA interference (RNAi). RNA interference refers to a unique defense mechanism through which small double stranded RNAs (dsRNA) silence cognate genes in a sequence specific manner. Since its invention it has become a powerful tool to suppress the expression or formation of certain proteins in the cell. Therefore in one embodiment, a dsRNA molecule that is homologous to a proinflammatory cytokine mRNA sequences is contemplated for use attached to an active nanoparticle of the present inventions for suppressing the activity of the target gene, in a preferred embodiment the target gene is expressed in a cell within or near an athersotic plaques. In comparison with conventional methods of drug treatment, this embodiment is contemplated to have a number of advantages in terms of long-term biological efficacy, specificity, with minimal side effects of the active nanoparticles of the present inventions. Even though the applications of siRNA to a large number of fields has increased exponentially over past few years, its role in understanding and preventing atherosclerosis is not known (Tang, et al., Acta Pharmacol. Sin. 2007, 28, (1), 1-9; herein incorporated by reference).

One of the primary concerns with using siRNA for biological application is providing a means of efficient delivery, without hampering its activity, directly to the site of action. It is contemplated that by administering intravenously dsRNA molecules without a delivery vehicle will allow ribonucleases to cleave the molecules leading to very low efficacy and short circulation time. Another key concern is the high negative charge present on the siRNA oligonucleotides, due to its phosphate backbone which would inhibit entry into the target cell. Efficient cell entry typically requires cationic particles, and the extent of cationic character of the delivery vehicle can be measured by N/P ratio where N represents positively charged species (typically amines) and P represents negatively charged species (the phosphate backbone). Excess positive charge can be toxic to a live cell and hence the N/P ratio value of 5-15 has been established to attain best transfection results (Kim, et al., J. Controlled Release 2008, 129, (2), 107-116; herein incorporated by reference). To overcome these issues many vectors for siRNA delivery have been developed including viral and non-viral vectors. Among them, the non-viral vectors offer better applicability due to low toxicity, ease of synthesis and low immune response over their viral counterparts (Zhang, et al., J. Controlled Release 2007, 123, (1), 1-10; Gao, et al., Mol. Pharmaceutics, ACS ASAP; herein incorporated by reference). Thus, the inventors contemplate additional embodiments comprising active nanoparticles of the present inventions further comprising attached non-viral vectors, such as cationic liposomes, peptides, and polymers (for examples of non-viral vectors, see, Zhang, et al., J. Controlled Release 2007, 123, (1), 1-10; herein incorporated by reference). In some embodiments, polymers include but are not limited to synthetic peptides (Mok, et al., Biopolymers 2008, 89, (10), 881-888; herein incorporated by reference) poly-L-lysine (PLL), (Kim, et al., Bioconjugate Chem. 2008, 19, (11), 2156-2162; herein incorporated by reference) and branched or linear polyethylenimine (PEI) (Kim, et al., J. Controlled Release 2008, 129, (2), 107-116; herein incorporated by reference). It is contemplated that the use of such polymers would provide desired circulation times (preferably increased over other types of siRNA delivery) and improved half-life of the siRNA particles for providing desired levels of treatment. Another critical aspect of successful siRNA treatment is effective release of the siRNA following endocytosis by the target cell. To ensure efficient release of the oligonucleotide, additional embodiments of the present inventions further comprise linkers, such as a disulfide linker (for providing S—S linkage) to covalently link a therapeutic to the active nanoparticles of the present inventions, for examples of linker molecules contemplated for use in the present inventions, see, Low, et al., Acc. Chem. Res. 2008, 41, (1), 120-129; all of which is herein incorporated by reference). siRNA was used in cancer related therapies (Jeong, et al., Bioconjugate Chem., ACS ASAP; herein incorporated by reference).

Statins and Statins are the best selling class of drugs in the world. As the inhibitors of 3-hydroxy-3-methylglutaryl-coenzyme A reductase, a key enzyme in cholesterol biosynthesis, statins are effective in the treatment of hypercholesterolemia related to cardiovascular disorders. Besides their lipid lowering activities, statins exhibit anti-inflammatory properties (Weitz-Schmidt, Trends Pharmacol. Sci. 2002, 23, 482-486; herein incorporated by reference). It is suggested that those effects are largely dependent on the enhancement of nitric oxide (NO) level (Napoli, et al., J. Nitric Oxide Biol. Chem. 2001, 5, 88-97; herein incorporated by reference), leading to a decrease in platelet activation, attenuation of adhesion molecules expression, and a decrease in inflammatory cytokine production (Mason, et al., Circulation 2004, 109 (Suppl 1), II34-II41; herein incorporated by reference).

Moreover, the statin-mediated inhibition of vascular endothelial growth factor synthesis, the major angiogenic factor, may contribute to the attenuation of angiogenesis.

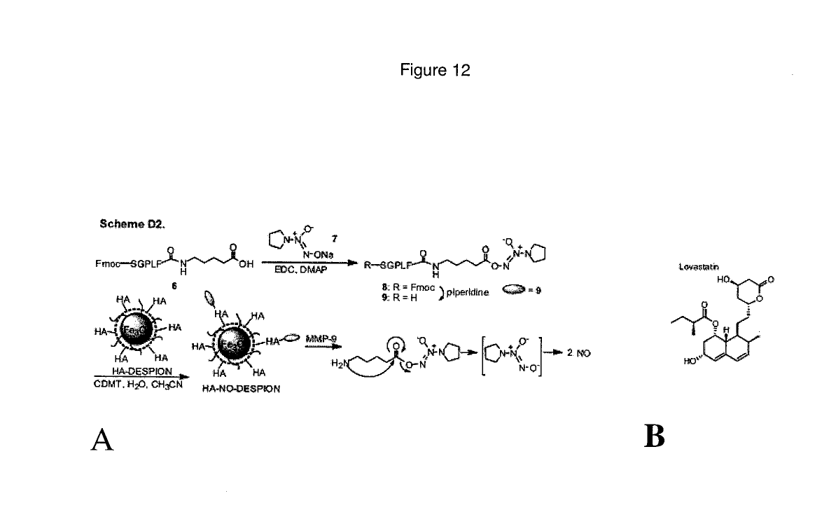

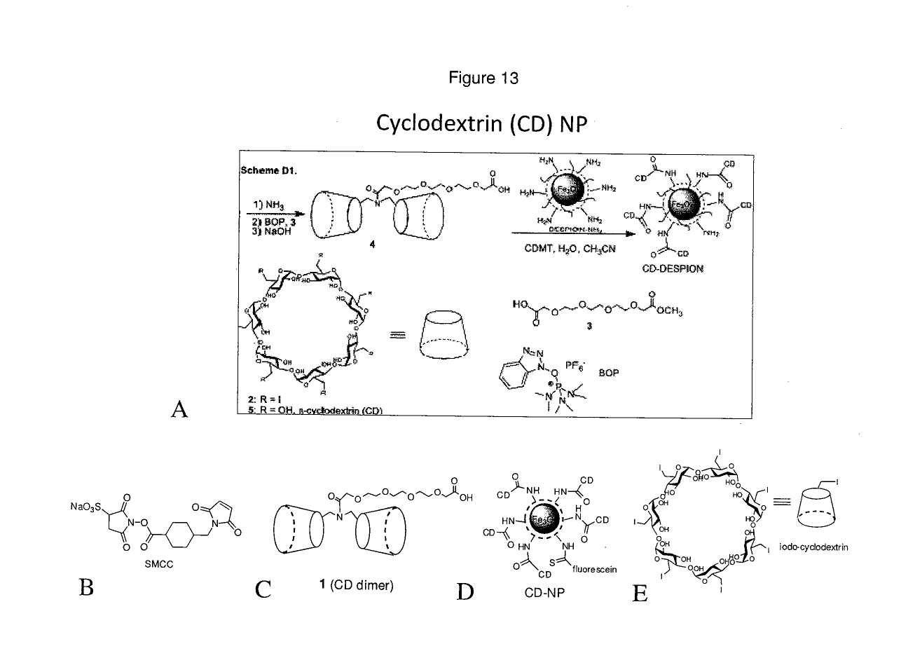

Recently, statins such as lovastatin may also physically dissolve part of the cholesterol crystals in the plaques, reducing the possibility of sharp cholesterol crystals puncturing the fibrous cap. A targeting compound of the present inventions comprising lovastatin is contemplated for use in treating cholesterol plaques, see, FIG. 12.

The innovation of this proposed study lies in the development of a magnetic NP based technology to target two molecular markers, i.e., CD44 and cholesterol, for plaque imaging. Despite their important in plaque biology, CD44 and cholesterol were not investigated previously for molecular imaging. Monitoring these markers can potentially allow inflammation assessment and for observing plaque development, which is crucial for early detection and plaque treatment. NPs present a powerful platform that can be designed and engineered to achieve desired biodistribution, targeting and imaging. Successful completion of this study can not only enhance our fundamental understanding of how NPs interact with the biological systems, but also provide more comprehensive evaluation of plaques as well as new targets for therapeutic development. In additional contemplated embodiments, polyethylene glycol (PEG) will be attached to these HA-NPs. It is known that PEG can significantly reduce non-specific cellular uptake of NPs by cells including macrophages, 47 which can improve their blood circulation time and biocompatibility and reduce non-specific uptake. Longer plasma half-life can favor NP accumulation in atherosclerotic plaques due to the enhanced endothelial permeability resulting from inflammation. The optimal construct with minimum non-specific uptake while maintaining HA-CD44 recognition will be identified through ELISA and flow cytometry analysis of cellular uptake.

Moreover, as CD44 is present on vascular endothelial cell surface and cholesterol crystal can also exist on plaque surface, they can directly bind with the probes in the lumen. This will enable the reduction in the dose of nanoprobes required for treatment and imaging without prolonged delay after probe administration, which will be important for future clinical translation and patient compliance. Additionally, the ability to non-invasively follow the development of atherosclerotic plaques and detect stages of plaque development can greatly benefit cardiovascular disease studies.

Other NP constructs are contemplated in additional embodiments, including zinc doped magnetic NP50 and FePt, 51 which have been shown to have greater relaxivities than magnetite NPs. The surface of these NPs will be decorated with targeting agents and PEGylated.

In order to determine the biodistribution of our nanoprobes, hydrazide groups will be attached to the NPs and couple 18F labeled 2-deoxy glucose (18FDG) to the antiCD44 NP through reductive amination. This is a very rapid reaction, compatible with the short half life of 18F (T1/2=110 minutes). The bio-distribution and clearance of the NPs inside the rabbits will be analyzed by PET/CT imaging. This will allow us to assess the whole body distribution and semi-quantify the amount of labels at plaque sites within one day of probe injection. Although the PET images have lower resolution, these images will be co-registered with high resolution CT measurements obtained concurrently on a fusion PET/CT scanner and compared with MR images. This multimodal imaging approach will help confirm plaque locations.

With the non-invasive nature of MRI, the plaque development process will be continuously monitored using antiCD44-NPs. Furthermore, plaque rupture will be triggered in animals with more advanced diseases and histology analysis will be performed to establish the correlations between MRI images and CD44 levels, characteristics of the atherosclerotic lesions, as well as rupture sites. A significant issue in the development of new nanomaterials for biomedical applications is their toxicity. As mentioned above, HA-DESPIONs are not toxic to cells and we did not observe any toxicity to the rabbits. Targeting cell surface receptors also enabled us to use much lower dosage in vivo compared to literature.

### EXPERIMENTAL

The following examples are provided in order to demonstrate and further Illustrate certain preferred embodiments and aspects of the present invention and are not to be construed as limiting the scope thereof.

In the experimental disclosure which follows, the following abbreviations apply: M (molar); mM (millimolar); μM (micromolar); nM (nanomolar); mol (moles); mmol (millimole); μmol (micromole); nmol (nanomole); gm (gram); mg (milligram); μg (microgram); pg (picogram); L (liter); ml (milliliter); μl (microliter); cm (centimeter); mm (millimeter); μm (micrometer); nm (nanometer); ° C. (degrees Centigrade or Celsius), s (second), h and hr (hour), and wk (week).

### Example I

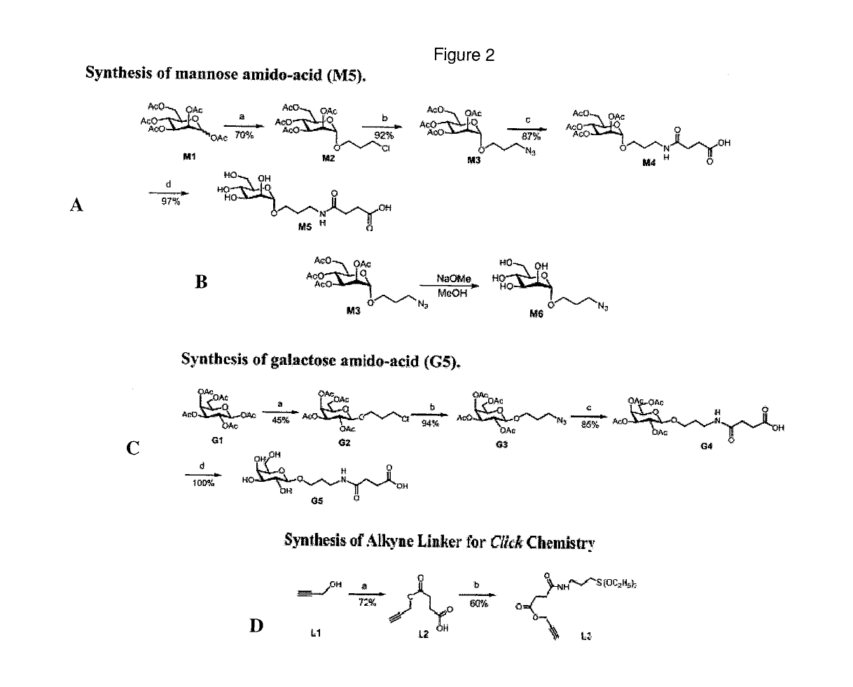

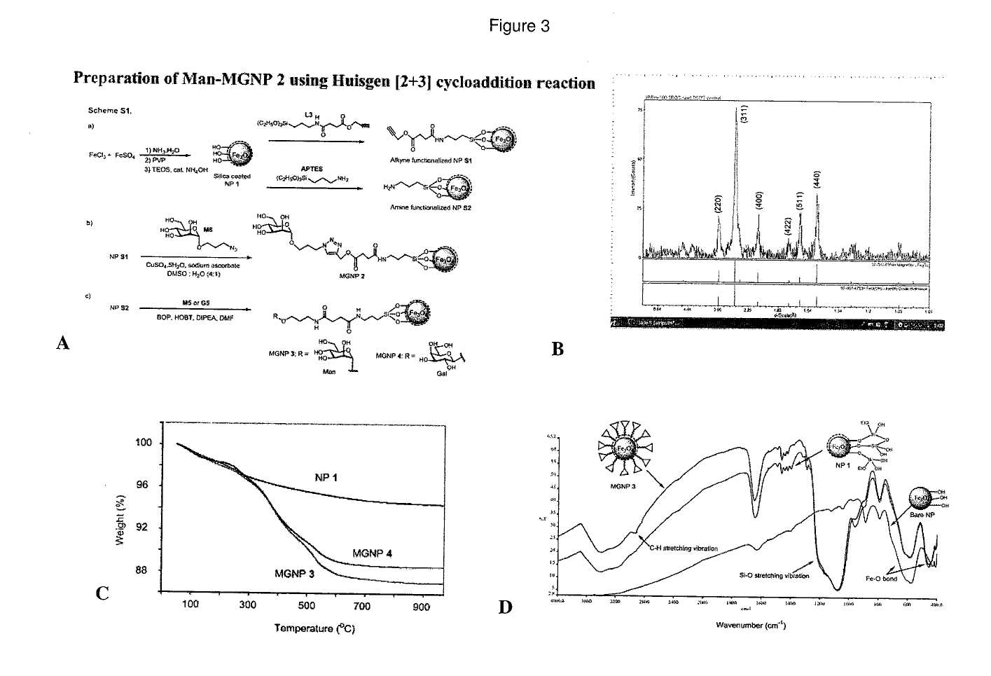

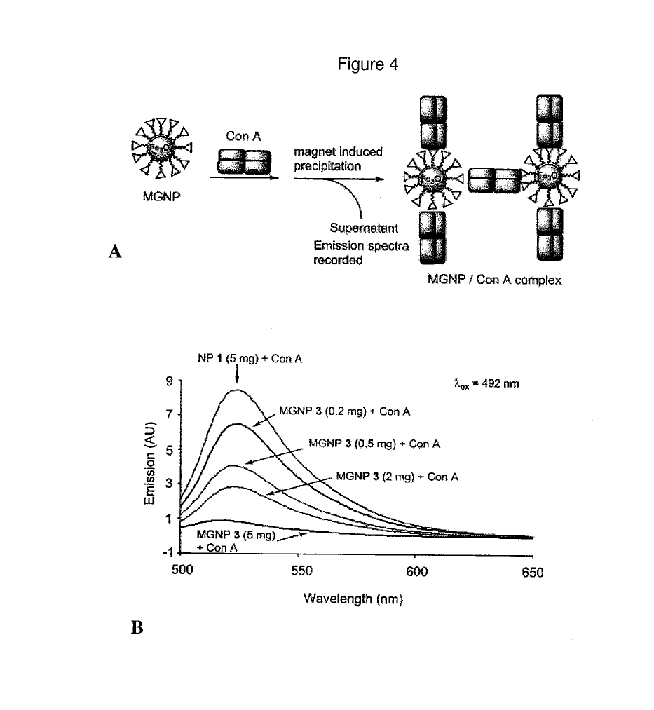

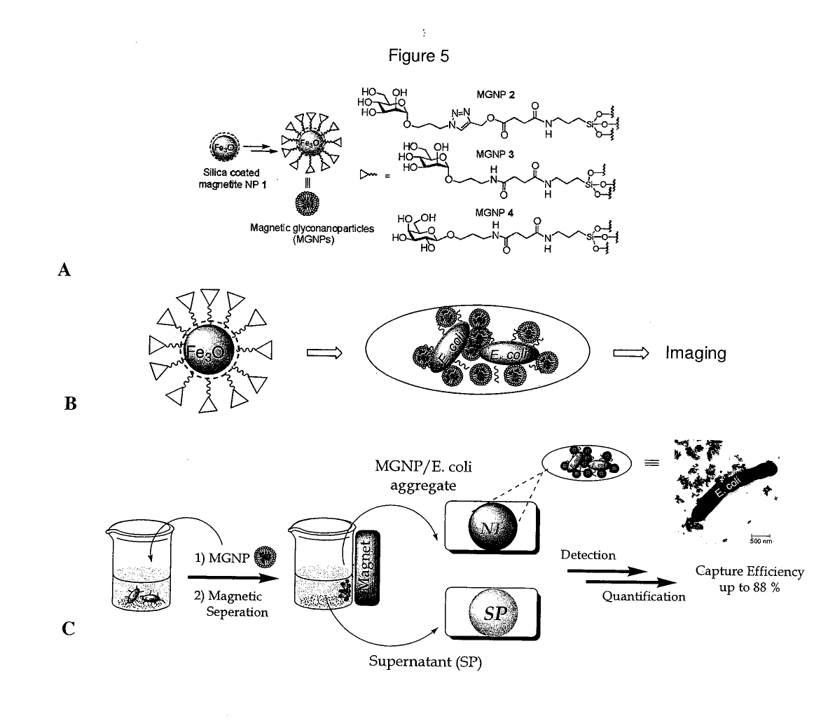

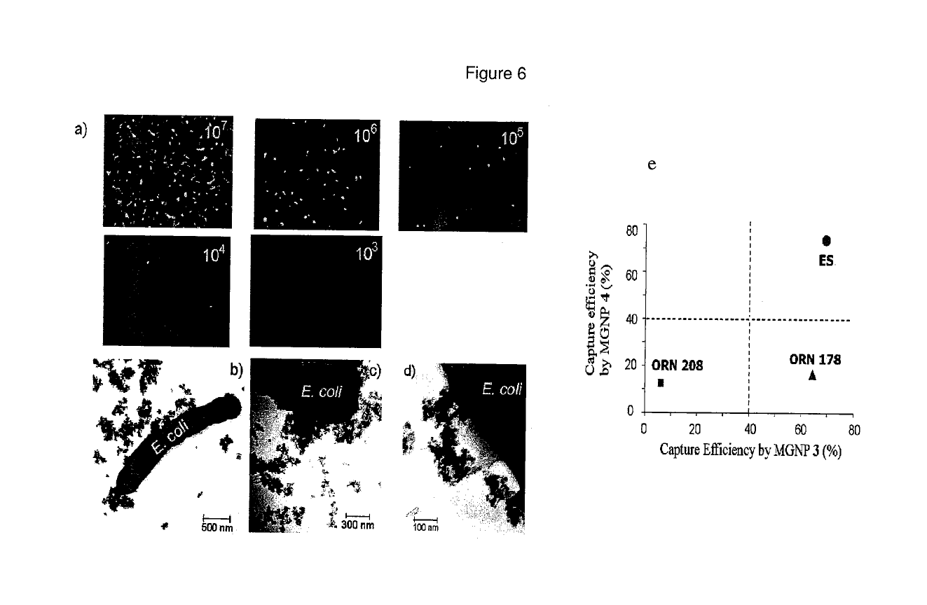

This example demonstrates exemplary carbohydrate synthesis, nanoparticle construction, nanoparticle characterization techniques, use of nanoparticles of some embodiments of the present inventions for binding to a cell in vitro, cellular specificity, etc. (El-Boubbou, et al.; J. Am. Chem. Soc. 2007, 129, 13392-13393; and online supplement; all of which are herein incorporated by reference) in part shown in exemplary FIGS. 2-6.

### Example II

This example demonstrates exemplary synthesis of carbohydrates and nanoparticles for contemplated use of these nanoparticles in some embodiments of the present inventions.

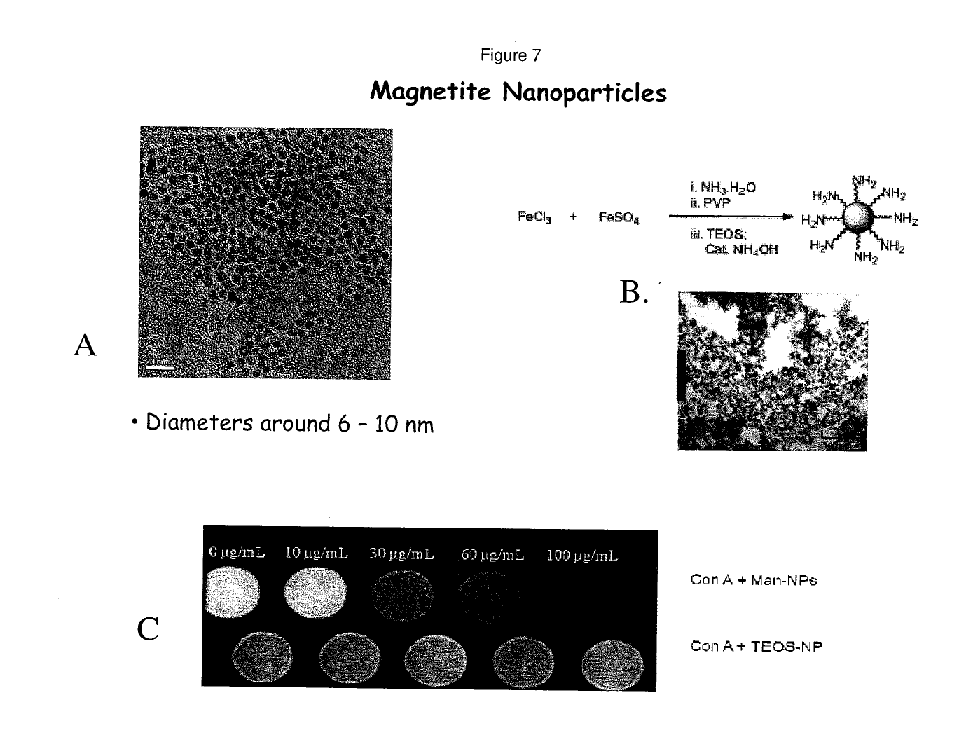

For this purpose, concanavalin A (ConA) and mannose were selected as a lectin-carbohydrate pair, where mannose coated nanoparticles (Man-NP) (FIG. 7A and B) were employed to detect the presence of the lectin. TEOS nanoparticles were conjugated to a linker carrying mannose moiety, incubated with varying concentrations of ConA overnight, and the variation of T2 relaxation due to surface binding was measured. Cluster formation of these nanoparticles, upon incubation with ConA, resulted in a quick and significant decrease in the spin-spin relaxation time T2 of the neighboring water molecules in the medium. A decrease in brightness of the MR image of the samples was observed as the concentration of Con A was increased (FIG. 7C), which is attributed to a decrease in T2 relaxation time. In fact, MGNP-ConA aggregates resulted in a rapid precipitation that was noticeably detected by the naked eye. Similar behavior was not observed when unmodified TEOS nanoparticles (TEOS-NP) without mannose were incubated with ConA (FIG. 7c), thereby indicating that the precipitation of nanoparticles is due to a specific binding event of the lectin and its carbohydrate receptor, thus illustrating the utility of magnetic nanoparticles as MRI contrast agents. Having established the magnetic properties of the nanoparticles, the next study was to establish the ability of NPs of the present inventions to target the atherosclerotic plaque.

### Example III

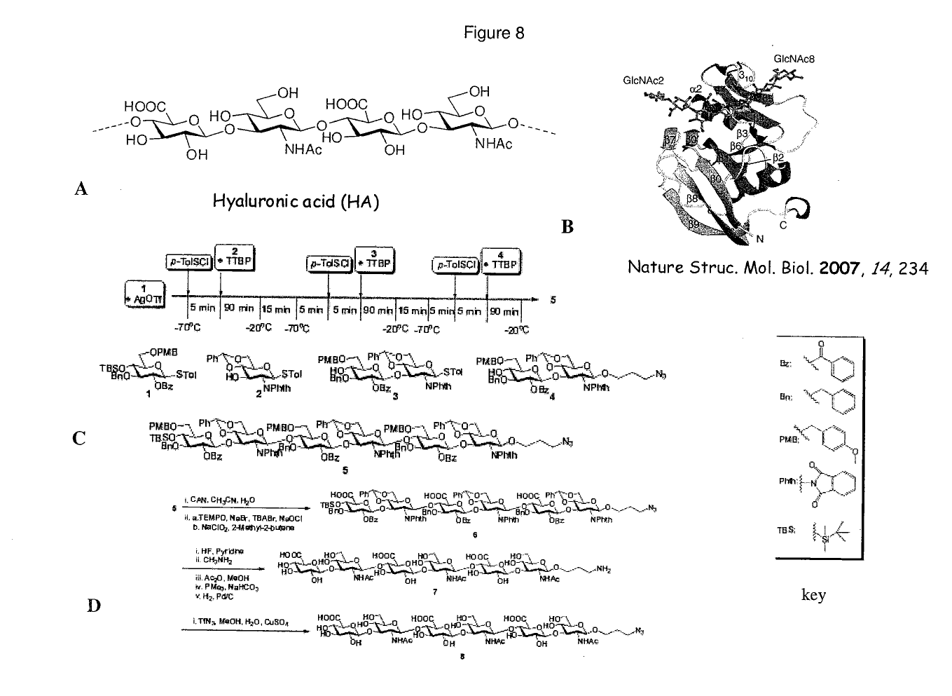

This example demonstrates exemplary synthesis of Hyaluronic Acid (FIG. 8A) Oligosaccharides for attaching to nanoparticles of the present inventions. Design, synthesis and characterization of hyaluronic acid coated magnetic nanoparticles: HA oligosaccharides (sHA) of different length will be assembled using the one pot pre-activation glycosylation technique developed by the inventors (Huang, et al., Chem. Eur. J. 2007, 13, (2), 529-540; herein incorporated by reference).

In vitro studies to determine the efficacy of the HA coated magnetic nanoparticles were performed using the commercially available high molecular weight HA, for the in vivo studies, nanoparticles were contemplated to compete with native HA for binding to CD44 (FIG. 8B). Therefore, the inventors synthesized smaller sHA than available commercially, for obtaining better binding properties and improving the ability of the nanoparticles to replace the native HA bound to CD44 at the site of lesion. The minimum length of sHA required to replace native HA was estimated to be six saccharide units long (Takeda, et al., J. Biol. Chem. 2006, 281, (52), 40089-40095; Sun, et al., Adv. Drug Delivery Rev. 2008, 60, (11), 1252-1265; herein incorporated by reference).

The synthesis of HA oligosaccharides ended with a deproteineated hexasaccharide (FIG. 8D), and was accomplished via two approaches, chemical (FIG. 8C) and enzymatic, allowing comparison of their efficiency as targeting ligands for binding to target CD44. Chemical synthesis will be further modified to obtain structural analogs of the naturally occurring oligosaccharides, to improve the binding properties of the naturally occurring HA oligosaccharides. Chemical synthesis of HA: Traditional approach to chemical synthesis of sHA involves two general pathways (Huang, et al., Chem. Eur. J. 2007, 13, (2), 529-540; herein incorporated by reference).

In the first method, glucuronic acid building blocks were directly used to react with glucosamine derivative. The homologation of the disaccharide can then be achieved by repeating selective protection-deprotection sequences followed by glycosylation to obtain different lengths of sHA oligomers. Due to low reactivity of glucuronic acid derivatives, yield for this synthetic approach is typically low. This was circumvented by using the corresponding glucose derivatives and adjusting the oxidation state at the end of synthesis of oligomers. Despite the modifications in synthetic strategies, the stepwise nature of this synthesis makes the whole process extremely cumbersome and low yielding. Recently Huang group has developed a new pre-activation based iterative one-pot oligosaccharide synthesis method, where multiple sequential glycosylations can be carried out in the same reaction vessel without intermediate purification, modifications, allowing construction of complex oligosaccharides in a timely manner (Huang, et al., Chem. Eur. J. 2007, 13, (2), 529-540; herein incorporated by reference).

This approach was implemented in synthesis of sHA resulting in quick assembly of tetra-, hexasaccharide. Simple deprotection steps yield pure, well-characterized oligomers. Specifically, as shown in FIG. 8 the hexasaccharide 5 was synthesized using building blocks 1, 2, 3, and 4 following the pre-activation based one-pot method (Scheme 1). Pre-activation of donor 1 was achieved by p-toluoyl sulfonyl triflate (p-TolSOTf) at −70° C. followed by glycosylation with acceptor 2 and tri-t-butyl pymidine (TTBP). TTBP helps to neutralize the triflic acid generated during the glycosylation. The reaction was then warmed up to −20° C. to destroy the unreacted activated donor. The newly formed disaccharide was then activated in similar fashion followed by addition of acceptor 3 and subsequently acceptor 4 to obtain hexasaccharide 5 within just a few hours. The final product is then purified by chromatography and characterized using NMR and high resolution mass spectrometry. Deprotection sequence began with removal of PMB groups, followed by two-step one pot oxidation using combination of TEMPO/NaOCl and NaClO2 to yield the tricarboxylic acid 6. TBS, phthalimido and benzoate groups were then removed using standard protocol followed by formation of acetamide. Staudinger reduction followed by hydrogenolysis then yielded compound 7. Terminal azide was regenerated using diazo transfer to obtain 8, which can be used in future to conjugate the sHA to nanoparticles. Having synthesized the naturally occurring version of sHA hexasaccharide, now the Huang group is working on synthesizing the structural analogs and screening them to determine the binding constant to CD44. The compounds with better binding affinity will be chosen to coat the nanoparticles.

Enzymatic synthesis of HA: Alternatively, the synthesis of sHA oligosaccharides has been accomplished via enzymatic degradation of the high molecular weight HA. In a typical experiment, HA (500 mg) was dissolved at a concentration of 3.33 mg/ml (150 ml total) in digest buffer (0.15 M NaCl, 0.1 M Na-acetate, adjusted to pH 5.2 with glacial acetic acid) and incubated at 37° C. for 30 min., followed by addition of bovine testicular hyaluronidase (4 mg in 40 ml buffer), and incubation at 37° C. for 3 days. The digestion was quenched by bringing the reaction mixture to boil. The hexasaccharide and tetrasaccharide products were isolated and purified by column chromatography.

### Example IV

This Example describes exemplary construction of magnetic nanoparticles for use in embodiments of the present inventions.

Synthesized magnetic nanoparticles were made coated with various high affinity ligands targeting atherosclerotic plaques. Synthesized and characterized magnetic NPs were immobilized with fluorophores and carbohydrates for pathogen detection and decontamination (El-boubbou, et al., J. Am. Chem. Soc. 2007, 129, 13392-13393; herein incorporated by reference), see Example I for an example of nanoparticle production.

Two iron sources FeCl₃ and FeSO₄ (molar ratio of Fe³⁺:Fe²⁺=1.0:0.5) were coprecipitated in the presence of NH₄OH as the base. The final pH of the reaction was then adjusted to approximately 11 to obtain nanoparticles about 10-20 nm in size which was then confirmed by TEM. In order to better suit bio-recognition purposes, the particles were then treated with polyvinylpyrrolidone (PVP) as surfactant and tetraethoxysilane (TEOS) as stable coated silica shell (Laurent, et al., Chem. Rev. 2008, 108, (6), 2064-2110; herein incorporated by reference).

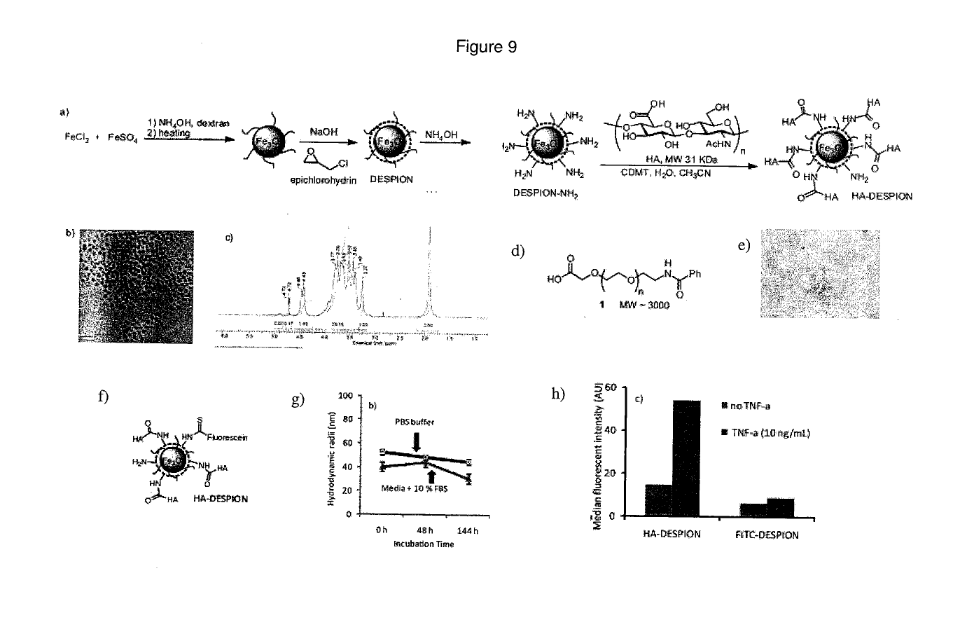

For MRI applications, highly colloidal water soluble SPIONs were prepared. This was achieved by the cold gel formation process by first neutralizing iron salts (Fe²⁺ and Fe³⁺) with NH₄OH in the presence of 10 kDa dextran at 0° C. to form paramagnetic gel, which was followed by heating to generate a SPION. The dextran coating on SPIONs was cross-linked with epichlorohydrin to yield dextran-epichlorohydrin SPIONs (DESPIONs), and treated with ammonia affording DESPION-NH₂ (left half of FIG. 9a).

The DESPION-NH₂ particles were highly colloidal and mono-dispersed, thus suitable for MRI applications. With the external amino groups attached to the DESPION-NH₂, a variety of targeting molecules were immobilized onto the NP. The first compound examined was HA. 2-Chloro-4,6-dimethoxy-1,3,5-triazine (CDMT) mediated amide coupling was made between HA (MW˜31 kDa) and DESPION-NH₂ that attached HA onto the NP surface (right half of FIG. 9a). The NPs were thoroughly characterized by a variety of techniques including X-ray diffraction, infra-red spectroscopy, TEM, themogravimetric analysis (TGA) and nuclear magnetic resonance (NMR). TEM images showed that the HA-DESPIONs were highly mono-dispersed with the average core diameter of 6 nm (FIG. 9b).

TGA analysis demonstrated that 80% of the particle weight was from the combined dextran and HA coating. NMR analysis of superparamagnetic particles was traditionally difficult due to the extreme line broadening caused by paramagnetic particles.

Recently, high resolution-magic angle spinning (HR-MAS) NMR was found to be a superior tool to investigate paramagnetic particles (Polito, et al., J. Am. Chem. Soc. 2008, 130, 12712-24; herein incorporated by reference HR-MAS NMR spectrum of the HA-DESPION of the present inventions gave solution like resolution (FIG. 9c), with the ratio of dextran to HA determined to be 1:3. With the thick layer of HA coating, it is most likely dextran is completely shielded by the HA, thus not interfering with the desired biological recognition of NP through HA.

Alternatively, the procedure was modified to prepare highly colloidal, biocompatible SPIOs that are similar to commercially available Feridex. Typically, the synthesis of a colloidal superparamagnetic iron oxides is achieved by the cold gel formation process involving two steps: The neutralization of iron salts (Fe 2+ and Fe 3+) with base (NH4OH) at 0° C. to form a weakly magnetic or paramagnetic gel, followed by, heating to convert the paramagnetic gel to a superparamagnetic colloid. The slurry formed is termed “paramagnetic” because of the large amounts of paramagnetic iron it has incorporated, but it exhibits no discrete magnetic properties such as attraction to an external, hand held magnet. The newly devised nanoparticles were composed of 5-10 nm magnetic iron oxide core, enriched with a 10 kDa dextran coating. To develop more stable and amino-functionalized nanoparticles, the dextran coating was cross-linked with epichlorohydrin to yield dextran-epichlorohydrin SPIONs (DESPIONs), and then treated with ammonia to provide functional amino groups on the surface affording DESPIO-NH₂. Amino groups can then react with the acid functionality of hyaluronic acid (HA) allowing attachment of a range of hyaluronan-bearing biomolecules. This afforded sHA-DESPIONs conjugates with unique biological properties. Moreover, sHA-HSPIONs were also fabricated where 16 KDa of hyaluronan polymer were directly incorporated on the surface of the nanoparticles with no dextran incorporation. TEM was used to confirm particle size and morphology of the monodispersed sHA-DESPIONs where the average diameter of the nanoparticles was found to be around 10 nm. Followed by evaluation of nanoparticles as MRI contrast agents.

### Example V

This Example describes the manufacture of exemplary HA magnetic nanoparticles of the present inventions for targeting CD44.

In order to target CD44, a particle was made by HA immobilization onto magnetic NPs. Specifically, an amide coupling was made between HA (MW˜31 kDa) and amines on the highly colloidal water soluble dextran coated magnetite NPs (DESPION) for HA-DESPION (FIG. 9a). To facilitate optical detection in addition to MRI, the fluorophore fluorescein isocyanate (FITC) was also added onto the NP surface through the thiourea bond formation with the residual amines (FIG. 9f). The NPs were thoroughly characterized by a variety of techniques including X-ray diffraction, infra-red spectroscopy, transmission electron microscopy (TEM), dynamic light scattering (DLS), themogravimetric analysis (TGA), zeta potential analysis and nuclear magnetic resonance (NMR). HA-DESPIONs were highly mono-dispersed with the average core diameter of 6 nm as shown by TEM (FIG. 9b) and a hydrodynamic radius of 50 nm determined through DLS. NMR studies of superparamagnetic particles have been traditionally difficult due to the extreme line broadening caused by paramagnetic particles. Recently, high resolution-magic angle spinning (HR-MAS) NMR was found to be a superior tool to investigate paramagnetic particles (Polito, et al., NMR Spectroscopy J. Am. Chem. Soc. 12712-24, 2008). HR-MAS NMR spectrum of our HA-DESPION gave solution like resolution with accurate integrations and coupling constants allowing us to quantify the ratio of surface ligands. The HA-DESPIONs have high relaxivities, with a R₂ value of 361 mM⁻¹ s⁻¹ at 3 T, which is higher than Feridex, a magnetic NP approved by FDA for MR imaging of liver (R₂=180 mM⁻¹ s⁻¹ at 3 T).

Colloidal stability under physiological conditions is an important factor for biological applications. The particle stability in biological media was evaluated by monitoring their hydrodynamic radii by DLS. The HA-DESPIONs maintained their sizes in both PBS buffer and culture media containing 10% fetal bovine serum (FBS) over one week, thus demonstrating their excellent colloidal stabilities (FIG. 9g). HA-DESPIONs are biocompatible, as MTT assay showed that they did not affect viabilities of several cell lines.

Next it was determined whether HA immobilized on NPs maintains its biological recognition. This was first tested through an enzyme linked immunosorbent assay (ELISA) with IgG-human CD44 chimera immobilized on the microtiter plate. HA-DESPION inhibited the binding between biotinylated HA polymer (0.5 microg/mL) and immobilized CD44 with an IC₅₀ value of 0.5 microg/mL, while the DESPION without HA did not show any inhibition even at 50 microg/mL. The interactions between HA-DESPION and CD44 were also evaluated using a human vascular endothelial cell line EA.hy926 (Bouïs, et al., Angiogenesis 4:91-102, 2001) by flow cytometry. Although this cell line expressed CD44 as confirmed by immunostaining with anti-CD44 mAb MEM-85, without inflammatory signals such as TNF-alpha, it uptook little HA-DESPION (FIG. 9h). This indicated that at the quiescent cellular state the CD44 receptors on these cells have low affinities with HA-NPs. To mimic the inflammatory process common in atherosclerotic plaques, TNF-alpha. (10 ng/mL) was added to the culture media of the EA.hy926 cells. This transformed CD44 into an activated state, resulting in a five fold increase in HA-NP uptake (FIG. 9h). Even with short incubation time of 30 minutes, binding between HA-NP and activated EA.hy926 cells was observed. Moreover, the interactions was HA dependent as FITC-DESPIONs without HA coatings was not taken up significantly with or without TNF-alpha. (FIG. 9h). Besides endothelial cells, similar preferential uptake of HA-NP was observed with a human macrophage cell line THP-1 cells by flow cytometry and the uptake was confirmed by Prussian blue staining, a staining method specific for iron ions (FIG. 9e). HA-NP uptake was significantly reduced in the presence of anti-CD44 mAb MEM-85, a mAb known to compete with HA binding (Ahrens, et al., J. Invest. Dermatol. 116:93-101 2001). These results suggested that HA-DESPIONs maintained the high specific affinity with CD44 and HA-DESPIONs should be selectively taken and concentrated at inflammatory sites.

In an further experiment to find out how and where the HA-DESPIONs are taken up by cells, cells were contacted with HA-DESPIONs. HA-DESPIONs were discovered to be taken in by the cells then transported through the cytoplasm and nucleus using Fluorescein conjugated HA-DESPIONs for showing the location of the NPs; including the use of a Lysotracker red channel showing lysosomes; and a DAPI channel showing location of nucleus; with an overlay these channels for cellular locations with a LASER image of the cells. The presence of 10% FBS or BSA did not affect the uptake significantly. The uptake was also confirmed by another method, i.e., Prussian blue staining, a staining method specific for iron ions. Besides endothelial a cell, similar preferential uptake of HA-NP was observed with THP-1 cells. HA-NP uptake was significantly reduced in the presence of anti-CD44 mAb MEM-85, a mAb known to compete with HA binding. These results suggested that HA on NPs maintained its high affinity with CD44 and HA-DESPIONs should be selectively uptaken and concentrated at inflammatory sites. In additional contemplated embodiments, polyethylene glycol (PEG) will be attached to these HA-NPs.

### Example VI

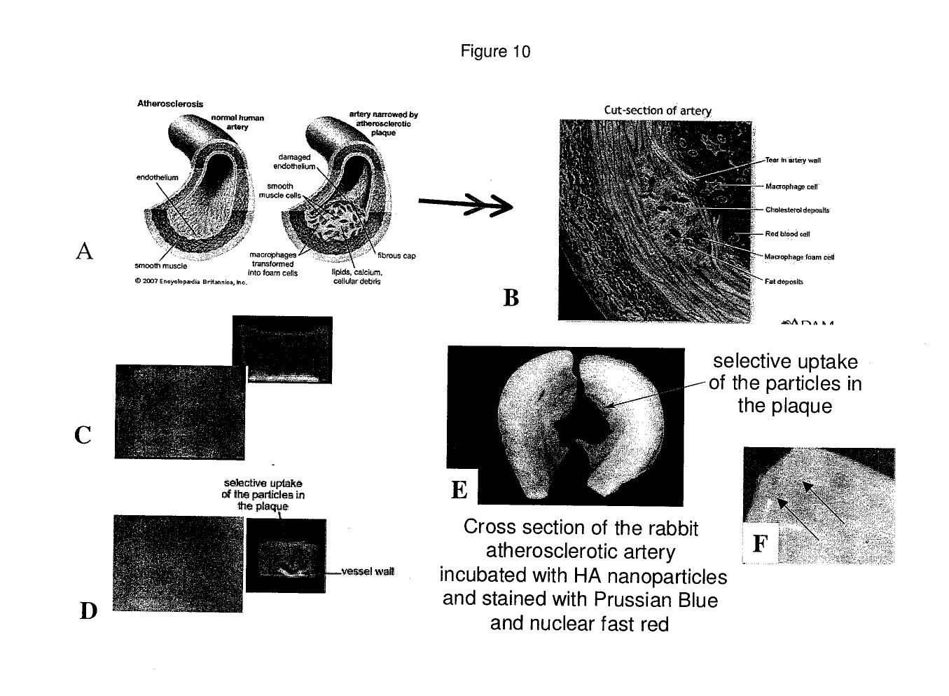

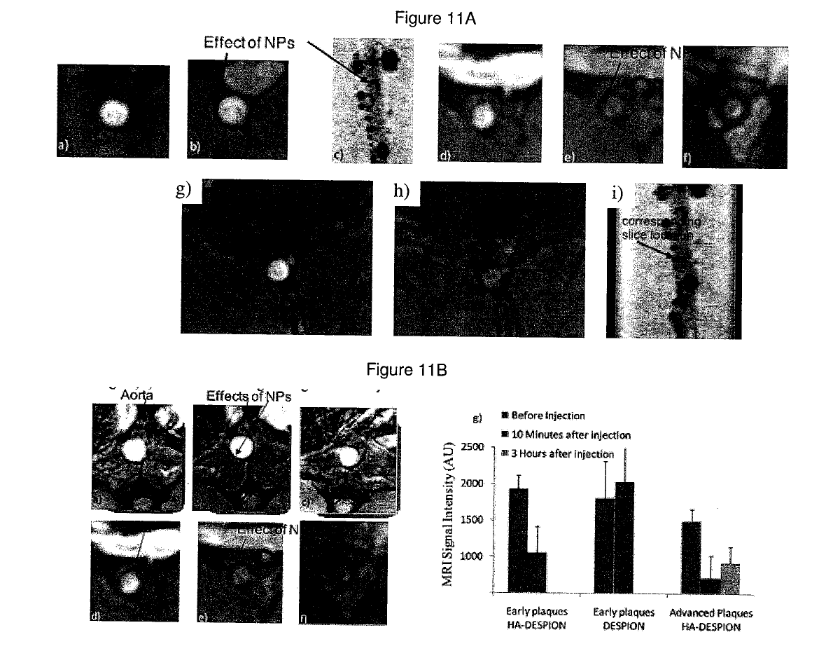

This Example demonstrates exemplary application of nanoparticles in atherosclerotic plaque targeting and imaging. Specifically, the use of exemplary HA magnetic nanoparticles of the present inventions for targeting activated human macrophage cells (FIG. 9e) and MRI imaging of atherosclerosis tissue (FIGS. 10 and 11) are shown.

In an exemplary targeting and imaging experiment, the atherosclerotic tissue and the healthy tissue were incubated with 0.5 mL suspension of HA coated magnetic nanoparticles (3 mg/mL) at 37° C. for 48 hr. After incubation, tissue was washed three times with PBS buffer and stained for iron using Prussian blue staining. In brief, the tissue was treated with 2:1 mixture of 2% potassium ferrocyanide and 2% hydrochloric acid (HCl) for 25 min, washed with PBS three times and counterstained with nuclear fast red. Tissue was given final PBS wash to remove the excess stain and observed under a microscope. The results (FIG. 10, D, E, F and FIG. 11b-c, e-f, and g-i) clearly indicate that the amount of nanoparticles adhering to the plaque tissue (FIG. 10D is significantly greater) as indicated by the Prussian blue stain, than the healthy tissue (FIG. 10C), confirming selective targeting the plaque using HA.

After demonstrating that HA-NPs can bind with CD44 as well as activated CD44 expressing cells, its selectivity in tissue binding was examined. HA-DESPIONs (0.05 mg Fe/mL) grams injected were incubated with both normal rabbit artery tissue and atherosclerotic artery tissues. The unbound NP was removed by thorough washing and the presence of the NPs in the tissues was detected by Prussian blue. The healthy rabbit artery tissue showed little Prussian blue staining, demonstrating that it did not retain much HA-DESPIONs (FIG. 10A). On the contrary, HA-DESPIONs bound to the atherosclerotic tissue strongly while the vessel wall remained largely unstained (FIG. 10C, D). Addition of high concentration of free HA (2 mg/mL) during HA-DESPION incubation completely abolished plaque binding. Furthermore, the DESPION without HA coating did not stain the atherosclerotic tissue, suggesting the HA-DESPION/plaque tissue interactions were due to specific binding with HA (FIG. 10F). The binding of HA-DESPION with plaque tissue was easily detected by MRI, as the selective darkening of the plaque was observed due to the adhesion of NPs to the tissue (FIG. 10E) while the darkening effect was absent with normal tissue (FIG. 10C). This indicated that HA-DESPION can be used to selectively target and image the atherosclerotic plaques.

As a model for early plaque formation, a rabbit's aorta was injured through a balloon-catheter procedure and then fed with high cholesterol diet for three weeks. The HA-DESPION (1 mg of Fe/kg of body weight corresponding to 0.04 mg Fe/mL of blood) was injected into the ear vein of the rabbit. Although little plaque developed at this stage with no stenosis (FIG. 11a), surprisingly the inventors observed selective darkening of the rabbit artery wall by MRI thirty minutes after HA-DESPION injection (FIG. 11b). The rabbit was sacrificed to have its aorta imaged ex vivo by MRI (FIG. 11c). The stain location correlated well with MR images. It is emphasized that the dosage in this study was much lower than the amount (56 mg Fe/kg) of SPION previously used for atherosclerotic plaque imaging of rabbit aortas (Sigovan, et al., Radiology 252:401-409, 200; Morris, et al., Arterioscler. Thromb. Vasc. Biol. 28:265-271, 200; Durand, et al., J. Vasc. Res. 44:119-128, 2007; and Ruehm, et al., Circulation 103:415-422, 2001) and imaging was performed without much delay after injection, which highlight the advantages of targeting plaque surface receptors. NPs were also tested on a rabbit with fully developed plaques after a balloon de-endothelialization process and six months of high cholesterol diet. Immediately after injection, selective darkening of the plaque areas was observed (FIG. 11d-f), which had a different profile compared with the images from the rabbit with early stage plaques (FIG. 11b). The HA-NPs were biocompatible as no toxicities on these rabbits were observed two months following NP injection.

The inventors contemplate injecting these HA magnetic nanoparticles into atherosclerotic mice for demonstrating efficacy for in vivo imaging. Tissue imaging studies: Since formation of the atherosclerotic plaque is marked by accumulation of HA, due to over-expression of the CD44 receptor, we propose to study the adhesion ability of the HA coated nanoparticles to atherosclerotic tissue. The tissue will be cut in 4 Nm wide sections and rehydrated in PBS before being treated with excess nanoparticles and incubated for 12-16 hrs. After incubation, the tissue will be washed with PBS and imaged using MRI to observe adhesion of the nanoparticles on the tissue surface. As control, pig artery tissue will be processed in similar fashion, as it does not express CD44.

Cell imaging studies: During the progression of atherosclerosis, the CD44 receptor is abundantly present not only on the endothelium but also on active macrophages recruited at the sight of lesion. Uptake of the HA nanoparticles will be studied in these macrophages to illustrate the role of CD44 receptor during pathogenesis of the disease. The human macrophage cell line U937 will be purchased from ATCC (cat. no. CRL-1593.2) and cultured according to instructions. The macrophages will be incubated for 48 hrs in medium containing 5% lipoprotein deficient serum and ox-LDL (50 Ng/mL, Biomedical technologies Inc, Stoughton, Mass., cat. no. BT 910). The cells will then be suspended in serum free RPMI media containing the previously optimized concentration of the nanoparticles, in 96 well plates at an approximate concentration of 1×10 4 cells per well. The cells will be incubated for 12-16 hrs in serum containing fresh media. The plate will be centrifuged to obtain the concentrated cell mass that will be subjected to MRI imaging studies to observe adhesion of the nanoparticles to the macrophage cell surface. tested the NPs on rabbits with advanced plaques. Selective darkening (52% signal reduction) of the plaque areas was observed ten minutes after injection (FIG. 11Be,g), with the signal reduction lasting more than three hours (FIG. 1B f,g). The area losing signals was much larger than that from the rabbit with early plaques, presumably due to more advanced plaque formation. Histology analysis is being performed to confirm the co-localization of HA-NP with CD44 and to correlate the MRI contrast changes with CD44 expression levels. The HA-NPs are biocompatible, as no toxicities on these rabbits were observed three months following NP administration.

Contemplated In vivo imaging studies: Eight-week-old male ApoE-deficient mice (C57BL/6) will be purchased from Jackson Laboratories (Bar Harbor, Me.), and maintained on an atherogenic diet (Research Diets, New Burnswick, N.J.), with subcutaneous angiotensin II infusion (Sigma, St. Louis, Mo.) at a dose of 0.75 mg/kg/day. Mice will be fed ad libitum and will have free access to water. After eight weeks, they will be divided into three groups: two controls and one study group. The study group will be injected with the HA coated nanoparticles suspended in saline solution, intravenously through the tail vein. One control group will be given Feridex (Berlex Laboratories) injection (1 mmol/kg iron, undiluted, injected over three minutes), and the other control group will be injected with dextran-coated nanoparticles suspended in saline solution, manufactured by our lab. MRI will be performed at 30 mins, 2 hr, 4 hr, and 24 hr intervals at the Department of Radiology, Michigan State University. Three days after the injection, the mice will be euthanized with CO₂ and the heart and aortas will be perfused under physiological pressure. The plaque area along with liver, lungs, kidney, spleen, and heart will be harvested, stained for iron using the Prussian blue staining protocol, uptake of the nanoparticles will be determined, and will be compared with the data obtained from MRI.

### Example VII

This example describes contemplated construction of magnetic NPs for drug delivery (FIG. 12).

Particles to be used in vivo: For the in vitro studies, the hexasaccharide carrying the azide will be conjugated to the nanoparticles via copper catalyzed click reaction. For this purpose, the nanoparticles will be conjugated to alkyne functionality via a PEG linker. Precisely, 4-pentanoic acid (9) will be activated using N-hydroxy succinimide (NHS, 10) and DCC to obtain 11 which will be characterized by NMR and mass spectrometry. The activated ester will be coupled to a heterobifunctional PEG (12, Sigma-Aldrich, cat. No 671487) in presence of triethyl amine to obtain 13 which will be purified by size exclusion chromatography and characterized using NMR and mass spectrometry. The acid moiety on 13 will be activated with NHS as previously described to give

Magnetic nanoparticles coated with HA with or without dextran incorporation were furnished, as described. The nanoparticles were characterized using TEM.

### Example VIII

The following examples demonstrate exemplary contemplated embodiments for treating patients using targeting nanoparticles of the present inventions, in particular for targeting cholesterol crystals (see, FIG. 1, C-E), using cyclodextrin coated nanoparticles.

In order to target cholesterol crystal, cyclodextrin (CD) dimers used for targeting were shown to be able to bind cholesterol (Breslow, et al., J. Am. Chem. Soc. 118:8495-8496, 1996). Dimerization of iodo-cyclodextrin with NH₃ followed by functionalization with a carboxylic acid will lead to CD dimer 1. It can then be immobilized onto the amine functionalized magnetic NPs generating CD-NP followed by fluorescence labeling with FITC. As there will be multiple CDs on the surface of the NPs, the avidity between the CD-NP and cholesterol crystal should be high due to the multivalent effect (Mammen, et al., Angew. Chem. Int. Ed. 37:2754-2794, 1998). The binding between cholesterol and CD-NP will be evaluated using a competition assay with radiolabeled cholesterol and varying concentrations of unlabeled cold cholesterol (Mast, et al., A J. Lipid Res. 46:1561-1568, 2005). The linker distance between the two CD units, the density of CD dimers on the NPs, magnetic relaxivity, colloidal stability, and biocompatibility will be examined to select the optimal NP constructs for cholesterol imaging.

The NP diameters can have a significant effect on imaging. Larger magnetic particles give better contrast per particle due to their higher iron content, while smaller particles have the capacity of deeper plaque tissue penetration. Bio-distribution of the particles is also dependent upon the particle sizes, with larger particles having shorter blood half-life (Lipinski, et al., J. Am. Coll. Card. 52:492-494, 2008). Magnetic NPs with various core diameters (e.g. 6, 30, 100 and 1000 nm) (Harris, et al., Glycobiology 18:638-648, 2008) and suitable targeting ligands will be synthesized, and their binding with CD44 and cholesterol will be evaluated as described above. The availability of various sizes of particles will enable us to tailor the probe. For example, to image the activated endothelium lining the atherosclerotic plaques expressing CD44, larger anti-CD44 HP (diameter approximately 1000 nm) will be tested. Even a single magnetic particle with 1 micrometer diameter would be observed by MRI (Reddy, et al., Exploiting Lymphatic Transport and Complement Activation in Nanoparticle Vaccines Nat. Biotechnol. 25:1159-1164, 2007). With their rapid clearance from blood, high contrast images of the plaque surface can be potentially obtained with short incubation time. On the other hand, to access the cholesterol laden plaque core, small CD-NPs (diameter approximately 20 nm) will be examined. These smaller particles should circulate in the blood much longer, which will have sufficient time to penetrate the endothelial lining and reach the lipid core.

### Example IX

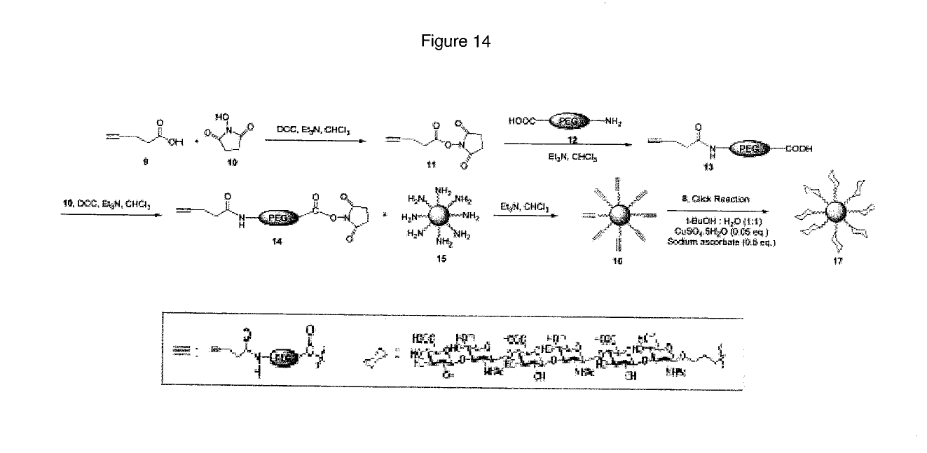

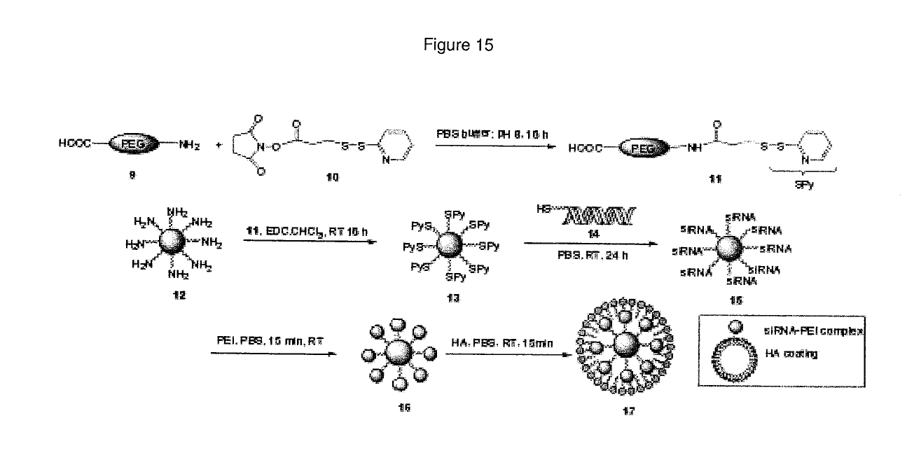

The following examples demonstrate exemplary embodiments for treating patients using targeting nanoparticles of the present inventions. This examples described a contemplated composition and method for an embodiment of the present inventions wherein an exemplary carbohydrate ligand is prepared (FIG. 14) and a targeting ligand is a covalently linked target ddsRNA (siRNA for TNF-alpha (FIG. 15) to magnetic nanoparticles via a disulfide bond and make it cationic by coupling to branched polyethylenimine (PEI). The dsRNA will be released only after endocytosis of the nanoparticles due to the reducing environment inside the endosome (Low, et al., Acc. Chem. Res. 2008, 41, (1), 120-129; herein incorporated by reference).

To optimize the dose concentration and minimize the loss of the particles to healthy cells, exemplary cell adhesion molecules as receptors for targeted delivery will be used that are abundantly present at the site of lesion (Haverslag, et al., Cardiovasc. Hematol. Disord.: Drug Targets 2008, 8, 252-260; herein incorporated by reference).

The siRNA carrying nanoparticles will be incubated with active human macrophages to study their ability to inhibit TNF-α secretion. They will also be injected intravenously into atherosclerotic mice to study their effect on the plaque growth and disease progression.

The following is a description of a design of the nanoparticles for targeted drug delivery: Design of the magnetic nanoparticles should fulfill a number of key requirements to exhibit the anticipated efficacy. These factors are discussed below.

For the purpose of in vitro studies, the human siRNA will be conjugated and for the in vivo studies mouse siRNA will be conjugated to the nanoparticles. The unreacted siRNA molecules will be removed by extensive dialysis (MWCO 10,000) against deionized and distilled water. The amount of conjugated siRNA will be determined using polyelectrolyte agarose gel electrophoresis (PAGE). In a typical experiment, 1 Ng of siRNA nanoparticles will be incubated with 10 mM glutathione for 2 hrs at 37° C. The cleaved siRNA will then be run on 2% agarose gel and visualized with ethidium bromide staining. The particle size and morphology will be observed using transmission electron microscopy (TEM). The hydrodynamic diameter will be evaluated using dynamic light scattering instrument (DLS). Stock solution of PEI (5 Ng/mL) in PBS will then be added drop by drop to a suspension of 15 in PBS at varying weight ratios and incubated for 15 min to obtain 16. After incubation, 16 will be coated with varying amounts of HA oligosaccharide and incubated for 15 min. Size and surface zeta potential value of 16 will be measured as a function of amount of HA by DLS. Cytotoxicity assay of HA coated magnetic nanoparticles: PC-3 cells (ATCC, Manassas, Va., cat. no.CRL-1435) will be seeded in 96 well plate at a density of 1×10 4 cells per well and grown for 24 hrs at 37° C. in accordance with ATCC instructions. The cells will then be incubated in serum free RPMI media (Sigma-Aldrich, St. Louis, Mo., cat. No. R 8758) containing the HA coated particles with and without siRNA at varying concentrations. The cells will then be cultured with serum containing fresh media for 2 days. Cell viability will be determined by the ‘cell counting kit-8’ (CCK-8, Dojindo Molecular Technologies, Rockville, Md., cat. no. CK04-13) cell viability assay, which measures mitochondrial dehydrogenase activity inside the cells (Lee, et al., Int. J. Pharm. 2008, 364, (1), 94-101; herein incorporated by reference). In a typical experiment, 10 NL of a CCK-8 solution is added to 100 NL of RPMI media in each well, the plate is incubated at 37° C. for 1-2 hrs, and the absorbance is measured at 450 nm using a plate reader. Higher absorbance will be an indication of larger volume of dead cells. Data obtained from the assay will be used to decide optimum concentration of the nanoparticles to be used during the in vivo and in vitro experiments.

### Example X

Synthesis and characterization of the nanoparticles for targeted drug delivery: Amine coated nanoparticles will be prepared from the Fe 2+ and Fe 3+ salts as described in the preliminary data section. These nanoparticles will be reacted with a bifunctional linker (11).

The linker will be obtained by conjugating PEG-3000 (9, 1 eq., Sigma-Aldrich, cat. No 671487) and N-Succinimidyl 3-(2-pyridyldithio)-propionate (SPDP, 10, 3.2 eq., Bachem Americas Inc, Torrance, Calif., cat. No. Q2535), dissolved in dimethyl sulfoxide (DMSO, 4 mL/100 mg of PEG), in presence of PBS buffer (4 mL/100 mg of PEG) for 2 hrs. (Scheme 3) (Lee, et al., J. Controlled Release 2005, 105, (1-2), 77-88; herein incorporated by reference The PEG-SPDP conjugate is then purified using size exclusion gel chromatography and will be characterized using NMR obtain pure 11. Linker 11 will be conjugated to the nanoparticles expressing amine groups using EDC mediated coupling chemistry to obtain 13. The product will be purified using ultrafiltration. The extent of SPDP incorporation will be determined by adding 50 mM dithiothreitol (DTT, Sigma-Aldrich, St. Louis, Mo.) solution (Lee, et al., J. Controlled Release 2005, 105, (1-2), 77-88; herein incorporated by reference This results in reduction of the disulfide bond to generate pyridine-2-thione species which can be detected by UV absorption at 343 nm. The amount of pyridine-2-thione released is directly proportional to the extent of PEG conjugation. This will then be confirmed by using thermogravimetric analysis (TGA). The well-characterized compound 13 will then be reacted with the sulfahydryl group of the siRNA 14 (Dharmcon Research, Lafayette, Colo.), at various ratios of magnetic nanoparticles to siRNA in PBS for 15 mins to give 15 (Kim, et al., Bioconjugate Chem. 2008, 19, (11), 2156-2162; herein incorporated by reference).

Contemplated In vitro studies: The in vitro studies to determine the efficacy of the siRNA carrying nanoparticles to achieve selective inhibition of secretion TNF-α will be performed on human macrophage cells. In a typical experiment, the macrophage cell culture prepared as described in the specific aim 2 section and will be incubated for 48 hrs in medium containing 5% lipoprotein deficient serum and ox-LDL (50 Ng/mL, Biomedical technologies Inc, Stoughton, Mass., cat. no. BT 910). Upon incubation, the cells will be divided into three groups: one study group and two control groups. The study group cells will then be suspended in serum free RPMI media containing the previously optimized concentration of the nanoparticles carrying siRNA, in 96 well plates at an approximate concentration of 1×10 4 cells per well. One control group will be incubated with nanoparticles expressing scrambled siRNA, and the other will be incubated with naked siRNA. The cells will be incubated for 24-48 hrs in serum containing fresh media. Towards the end of the incubation period, the culture medium will be centrifuged for 10 min at 400 g. The supernatant will be isolated and subjected to THF-α enzyme linked immunosorbant assay (ELISA) detection using TNF-α ELISA-kit (BD Biosciences, San Jose, Calif., cat. no. 550610) according to manufacturer's instructions and read at 450 nm. Inhibition of the TNF-α secretion in comparison with the control samples will be quantified. To ensure the uptake of particles via endocytosis mechanism, the macrophage cells will be pretreated with different endocytosis inhibitors before the treatment with nanoparticles. The inhibitors will include amantadine (0.1, 0.5, and 5 mg/mL), phenylarsine oxide (0.1, and 0.5 Ng/mL), cytochalasin D (0.1, 0.5, and 5 NM), and vinblastine (1, 5, and 50 NM) and will be purchased from Sigma-Aldrich, St Louis, Mo. Selective uptake via the CD44 receptor will be confirmed by inhibition of CD44 using mouse anti-human CD-44 antibody with a fluorescent dye (Hermes 3, ATCC) prior to the treatment with nanoparticles. In vivo studies: All experimental procedures will be approved by the Institutional Animal Care and Use Committee (IACUC). The mice will be prepared as described in the in vivo imaging studies section. After eight weeks, the mice will be divided into three groups: one study group and two control groups. The study group mice will be injected with nanoparticles carrying TNF-α siRNA, one control group mice will be injected with nanoparticles containing scrambled siRNA, and the second control group will be injected with naked siRNA through the tail vein on days 0, 4, 10, 18, and 28. The mice will be subjected to MRI at 30 min, 2 hr, 4 hr, and 24 hr intervals following the injections. The measurement of the atherosclerotic plaque will be done using the MRI data. Three days after the final treatment of siRNA formulation, mice will be euthanized with CO2 and the heart and aortas will be perfused under physiological pressure. The plaque area will be harvested, measured, and compared to the control group followed by histochemical staining for iron using Prussian blue and macrophages using Mac-2 staining protocol.

### Example XII

This examples described a contemplated composition and method for an embodiment of the present inventions wherein an exemplary targeting ligand is an substrate of an enzyme associated with an athersotic plaque, for example MMP-9. An exemplary MMP-9 substrate peptide is “SGPLF” (see, Laurent, et al., Chem. Rev. 2008, 108, (6), 2064-2110; herein incorporated by reference) 6 (FIG. 12) with its N-terminal still protected with Fmoc group and C-terminal linked with 5-amino pentanoic acid is contemplated for synthesis on solid phase and purified via HPLC following cleavage from resin. The carboxylic acid 6 is contemplated for use to etherify diazeniumdiolate 7 leading to molecule 8. Mild base promoted removal of the Fmoc group is contemplated to yield the free amine 9, for coupling to a HA-DESPION of the present inventions through amide formation with some of the free carboxylic acids on HA. Due to the large amount of HA on the particle surface, amide formation with amine 9 should not interfere significantly with the desired HA/CD44 interaction (binding).

In another embodiment, a method for converting a protein into a nitric oxide (NO) donor is contemplated for attaching to an active nanoparticle of the present inventions.

The amount of diazeniumdiolate (or NONOates) incorporated on the HA-NO-DESPION will be determined by HRMAS-NMR analysis was systematically varied. Upon binding of HA-NO-DESPION to the plaques, MMP-9 is contemplated to cleave off the diazeniumdiolate, which will decompose releasing NO. Lovastatin is contemplated to be linked in a similar manner as diazeniumdiolate. The discharge of NO and lovastatin from NPs in the presence of MMP-9 will be evaluated first in vitro then in vivo.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be within the scope of the following claims.

1. A composition, comprising a targeting ligand, wherein said targeting ligand targets a molecule associated with an atherosclerotic plaque, and a nanoparticle.

2. The composition of claim 1, wherein said targeting ligand is selected from the group consisting of hyaluronic acid, dextrin, cyclodextrin, mannose, a lectin siRNA, MMP-9 sensitive linker, MMP-9 substrate peptide SGPLF, statin, fibrin, NO prodrug, Simivastatin, selectins, CD44 and CD44 structural analogs.

3. The composition of claim 1, wherein said targeting ligand is selected from the group consisting of a hyaluronic acid (HA) molecule and a cyclodextrin (CD) dimer.

4. The composition of claim 1, wherein said molecule is a target analyte.

5. The composition of claim 4, wherein said target analyte is selected from the group consisting of CD44, Vascular Cell Adhesion Molecule-1, Inter-Cellular Adhesion Molecule-1, cholesterol, fibrin, MMP, and phosphatidyl serine.

6. The composition of claim 4, wherein said target analyte is selected from the group consisting of CD44 and cholesterol.

7. The composition of claim 1, further comprising a therapeutic agent.

8. The composition of claim 7, wherein said therapeutic agent is selected from the group consisting of a siRNA, an anti-proinflammatory agent, interleukins, growth factors, siRNA, MMP inhibitors, cholesterol binding molecules, cortisone steroids and a HA antiadhesive.

9. The composition of claim 7, wherein said therapeutic agent is selected from the group consisting of lovastatin, diazeniumdiolate, anti-TNF-α siRNA, interferon (IFN)-γ, a colony stimulating factors, and a MMP-9 sensitive linker.

10. The composition of claim 7, wherein said nanoparticle comprises a magnetic compound.

11. The composition of claim 10, wherein said magnetic compound is selected from the group consisting of Iron Oxide, Fe₃O₄, iron cobalt, gold and iron-platinum (FePt).

12. The composition of claim 11, wherein said nanoparticle further comprises a positron emission tomography (PET) label capable of providing a diagnostic computed tomography (CT) image, wherein said PET label is selected from the group consisting of PET tracer ⁶⁴Cu and 18F labeled 2-deoxy glucose (18FDG).

13. The composition of claim 1, wherein said nanoparticle further comprises a label capable of providing a nuclear magnetic resonance (NMR) image, wherein said compound is gadolinium.

14. The composition of claim 1, wherein said nanoparticle further comprises a label capable of providing a magnetic resonance spectroscopy (MRS) spectrum.

15. The composition of claim 1, wherein said nanoparticle has a diameter greater than 0 and less than or equal to 6 nm.

16. The composition of claim 1, wherein said nanoparticle further comprises a fluorohphore fluorescein isocyanate (FITC).

17. A method, comprising,
a) providing,
i) a composition, comprising a targeting ligand, wherein said targeting ligand targets a molecule associated with an atherosclerotic plaque, and a nanoparticle;
ii) a patient at risk for a cardiac event;
iii) an magnetic resonance imaging device; and
b) administering said composition to said patient for providing a diagnostic magnetic resonance image of an atherosclerotic plaque.

18. The method of claim 17, wherein said targeting ligand is selected from the group consisting of a hyaluronic acid (HA) molecule and a cyclodextrin (CD) dimer.

19. The method of claim 17, wherein said targeting ligand targets a target analyte.

20. The method of claim 18, wherein said hyaluronic acid (HA) molecule targets a plaque macrophage.

21. The method of claim 18, wherein said cyclodextrin (CD) dimer targets a cholesterol crystal.

22. The method of claim 17, wherein said nanoparticle further comprises a therapeutic agent for targeted drug delivery.

23. The method of claim 22, wherein said nanoparticle is capable of delivering the therapeutic agent to a plaque; wherein said therapeutic agent is selected from the group consisting of a siRNA, a MMP-9 sensitive linker, MMP-9 substrate peptide SGPLF, a statin, lovastatin, fibrin, a nitrous oxide (NO) prodrug, Simivastatin, selectins, CD44 structural analogs, anti-proinflammatory agent, interleukin, growth factor, siRNA of TNF-alpha, MMP inhibitor, cholesterol binding molecule, cortisone steroid and a HA antiadhesive.

24. The method of claim 17, wherein said targeting ligand targets a physiological event selected from the group consisting of macrophage activation, cholesterol crystal formation, angiogenesis, and apoptosis.

25. The method of claim 17, wherein said patient has atherosclerosis.

26. The method of claim 17, wherein said atherosclerotic plaque is selected from the group consisting of a pre-plaque, wherein a pre-plaque is a damaged artery wall, a vulnerable plaque, and a growing plaque.

27. The method of claim 17, wherein said nanoparticle further comprises the capability of providing a medical image, wherein said image is provided by devices selected from the group consisting of magnetic resonance spectroscopy (MRS), nuclear magnetic resonance imaging (NMR), multimodal imaging, fluorescent, positron emission tomography (PET) and computed tomography (CT).

28. The method of claim 17, wherein said administering comprises injecting 50-60 nmol/kg of said nanoparticle.

29. The method of claim 17, wherein said nanoparticle comprises a magnetic compound.

30. The method of claim 29, wherein said magnetic compound is selected from the group consisting of Iron Oxide, Fe₃O₄, iron cobalt, gold and iron-platinum (FePt).

31. The method of claim 17, wherein said nanoparticle further comprises a positron emission tomography (PET) label capable of providing a diagnostic computed tomography (CT) image, wherein said PET label is selected from the group consisting of PET tracer ⁶⁴Cu and 18F labeled 2-deoxy glucose (18FDG).
